(19) United States
(12) Patent Application Publication
(10) Pub. No.: US 2006/0147505 A1
(43) Pub. Date: Jul. 6, 2006

(54) Water-dispersible wet wipe having mixed solvent wetting composition

(76) Inventors: Richard W. Tanzer, Neenah, WI (US); Michael R. Lostocco, Appleton, WI (US); Kelly D. Branham, Woodstock, GA (US); W. Clayton Bunyard, De Pere, WI (US)

Correspondence Address: BRINKS HOFER GILSON & LIONE, P.O. BOX 10395, CHICAGO, IL, 60610, US

(21) Appl. No.: 11/027,555

(22) Filed: Dec. 30, 2004

(51) Int. Cl. A61K 8/81; A61K 9/70

(52) U.S. Cl. 424/443; 424/70.16

(57) ABSTRACT

A wet wipe contains fibrous material, a binder composition for binding the fibrous material into an integral web, and a wetting composition containing water, a salt, and at least about 10 wt % of an organic solvent. The binder composition contains an ionic copolymer. The wet wipe is not dispersible in the wetting composition, and is dispersible in water containing up to 200 ppm of one or more multivalent ions. The ionic copolymer may be the polymerization product of a vinyl-functional cationic monomer and at least one non-ionic vinyl monomer. The ionic copolymer may be the polymerization product of a vinyl-functional anionic monomer and at least one non-ionic vinyl monomer.

### BACKGROUND

For many years, the problem of disposability has plagued industries that provide disposable wipes for surface cleaning, household and institutional cleaning, and medical cleaning. It is often advantageous for these wipes to be wetted with a cleaning liquid, particularly with a liquid containing an organic solvent. However, wipes that have acceptable in-use strength in the presence of a cleaning liquid typically will not readily dissolve or disintegrate in water. Thus, these cleaning wipes must be disposed of as solid waste, rather than by flushing down a toilet. This is especially problematic for medical cleaning, as a used medical wipe must undergo special procedures for disposal of medical waste.

In addition, the ability of these products to disintegrate in a landfill can be limited because a large portion of the product components, which may well be biodegradable or photodegradable, are encapsulated in or bound together by plastic which degrades over a long period of time, if at all. Accordingly, if the plastic disintegrated in the presence of water, the internal components could degrade as a result of the rupture of the plastic encapsulation or binding.

Typically, a wet wipe contains a coherent fibrous substrate, which desirably possesses a number of characteristics, such as softness and flexibility. The fibrous substrate of the wipe is typically formed by wet or dry (air) laying a generally random plurality of fibers and joining them together to form a coherent web with a binder composition. Past binder compositions have preformed this function well. However, fibrous substrates comprising these compositions tended to be non-dispersible and present problems in typical household sanitation systems.

One class of dispersible binder compositions includes polymeric materials having inverse solubility in water. These binder compositions are insoluble in warm water, but are soluble in cold water, such as found in a toilet. It is well known that a number of polymers exhibit cloud points or inverse solubility properties in aqueous media. See, for example, U.S. Pat. No. 5,509,913.

Other dispersible binders include ion-sensitive binders, including polymers comprising acrylic acid and alkyl or aryl acrylates. See, for example, U.S. Pat. Nos. 5,312,883, 5,317,063 and 5,384,189, and European Pat. No. 608460A1. In U.S. Pat. No. 5,312,883, terpolymers are disclosed as suitable binders for flushable fibrous substrates. The disclosed acrylic acid-based terpolymers, which comprise partially neutralized acrylic acid, butyl acrylate and 2-ethylhexyl acrylate, are suitable binders for use in flushable fibrous substrates in some parts of the world. However, these acrylic acid-containing ion-sensitive polymers, when used as binders for personal care products such as wet wipes, typically have reduced initial sheet wettability, increased dry sheet stiffness, increased sheet stickiness, reduced binder composition sprayability and relatively high product cost.

There exists a need for dispersible products possessing softness, flexibility, three dimensionality, and resiliency; wicking and structural integrity in the presence of body fluids (including feces) at body temperature; and true fiber dispersion after toilet flushing so that product does not become entangled with tree roots or at bends in sewer pipes. Moreover, there is a need for flushable products having water-dispersibility in all areas of the world, including soft and hard water areas. There is also a need for water-dispersible binders that do not reduce wettability of product with which they are used and are sprayable for relatively easy and uniform application to and penetration into products. Furthermore, there is a need for disposable wipes that maintain structural integrity when wetted with cleaning liquids and used to clean surfaces, but that can also be disposed of through household sanitary systems. Such a product is needed at a reasonable cost without compromising product safety or raising environmental concerns.

### BRIEF SUMMARY

In an embodiment of the invention, there is provided a wet wipe, comprising a fibrous material, a binder composition for binding the fibrous material into an integral web, and a wetting composition comprising water, a salt, and at least about 10 wt % of an organic solvent. The binder composition comprises an ionic copolymer, is insoluble in the wetting composition, and is dispersible in water containing up to 200 ppm of one or more multivalent ions. The wet wipe may not be dispersible in the wetting composition, and may be dispersible in water containing up to 200 ppm of one or more multivalent ions.

In another embodiment of the invention, there is provided a method of making a wet wipe, comprising applying a binder composition comprising an ionic copolymer to fibrous material to form a fibrous substrate, and wetting the fibrous substrate with a wetting composition comprising water, a salt, and at least about 10 wt % of an organic solvent. The wet wipe is not dispersible in the wetting composition, and the wet wipe is dispersible in water containing up to 200 ppm Ca²⁺ and/or Mg²⁺ ions. The method may further comprise drying the fibrous substrate prior to wetting the fibrous substrate, and may yet further comprise packaging the fibrous substrate as a wipe after drying the fibrous substrate and prior to wetting the fibrous substrate.

These embodiments may further include wet wipes and methods of making the same, wherein the ionic copolymer is the polymerization product of a vinyl-functional cationic monomer and at least one non-ionic vinyl monomer. The vinyl-functional cationic monomer may be selected from the group consisting of [2-(acryloxy)ethyl]dimethyl ammonium chloride, [2-(methacryloxy)ethyl]dimethyl ammonium chloride, [2-(acryloxy)ethyl]trimethyl ammonium chloride, [2-(methacryloxy)ethyl]trimethyl ammonium chloride, (3-acrylamidopropyl)trimethyl ammonium chloride, N,N-diallyl-dimethyl ammonium chloride, [2-(acryloxy)ethyl]dimethylbenzyl ammonium chloride, and [2-(methacryloxy)ethyl]dimethylbenzyl ammonium chloride. The vinyl-functional cationic monomer may be a precursor monomer selected from the group consisting of vinylpyridine, dimethylaminoethyl acrylate and dimethylaminoethyl methacrylate, where the polymerization product has been treated with a quaternizing reagent.

These embodiments may further include wet wipes and methods of making the same, wherein the ionic copolymer has the structure: 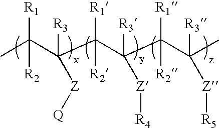
In this structure, x is from about 1 to about 15 mole percent, y is from about 60 to about 99 mole percent, and z is from about 0 to about 30 mole percent; Q is selected from C₁-C₄ alkyl ammonium, quaternary C₁-C₄ alkyl ammonium, and benzyl ammonium; Z, Z′ and Z″ are independently selected from —O—, —COO—, —OOC—, —CONH—, and —NHCO—; R₁, R₁′, R₁″, R₂, R₂′, R₂″, R₃, R₃′ and R₃″ are independently selected from hydrogen and methyl; R₄ is C₁-C₄ alkyl; and R₅ is selected from methyl, ethyl, butyl, ethylhexyl, decyl, dodecyl, hydroxyethyl, hydroxypropyl, polyoxyethylene, and polyoxypropylene.

These embodiments may further include wet wipes and methods of making the same, wherein the ionic copolymer has the structure: 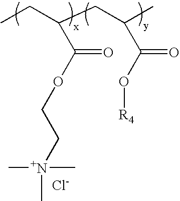
In this structure, x is from about 1 to about 15 mole percent, y is from about 85 to about 99 mole percent, and R₄ is C₁-C₄ alkyl.

These embodiments may further include wet wipes and methods of making the same, wherein the ionic copolymer is the polymerization product of at least one non-ionic vinyl monomer and a vinyl-functional anionic monomer selected from the group consisting of acrylic acid, methacrylic acid, itaconic acid, 2-acrylamido-2-methyl-1-propanesulfonic acid (AMPS), 2-methyl-2-propene sulfonic acid, vinyl sulfonic acid, styrene sulfonic acid, 2-sulfopropyl methacrylate, 3-sulfopropyl acrylate, and alkali salts thereof.

These embodiments may further include wet wipes and methods of making the same, wherein the ionic copolymer is the polymerization product of a vinyl-functional ionic monomer and at least one non-ionic vinyl monomer. The at least one non-ionic vinyl monomer may comprise at least one hydrophobic monomer with an alkyl side chains of 1 to 4 carbon atoms. The at least one non-ionic vinyl monomer may comprise at least one hydrophobic monomer selected from branched or linear alkyl vinyl ethers, vinyl esters, acrylamides, and acrylates.

These embodiments may further include wet wipes and methods of making the same, wherein the wetting composition comprises from about 0.3 wt % to about 10 wt % salt; wherein the salt is at least one member selected from the group consisting of NaCl, NaBr, KCl, NH₄Cl, Na₂SO₄, ZnCl₂, CaCl₂, MgCl₂, MgSO₄, NaNO₃, and NaSO₄CH₃; wherein the wetting composition comprises from about 10 wt % to about 75 wt % organic solvent; wherein the organic solvent is selected from the group consisting of alcohols, ketones, ethers, acetates, amines, amides, sulfides, sulfoxides, and halogenated hydrocarbons; wherein the organic solvent is selected from the group consisting of ethanol, iso-propanol, and acetone.

### BRIEF DESCRIPTION OF THE DRAWINGS

FIG. 1 is a graph of machine direction wet tensile strength (MDWT) of wet wipes as a function of salt concentration and organic solvent in the wetting composition;

FIG. 2 is a graph of MDWT of wet wipes for two different types of binder compositions, and for 4 different types of wetting compositions;

FIG. 3 is a graph of MDWT of wet wipes as a function of ethanol concentration in the wetting composition;

FIG. 4 is a graph of MDWT of wet wipes as a function of salt concentration and soaking time in hard water; and

FIG. 5 is a graph of MDWT of wet wipes as a function of wetting composition add-on and soaking time in hard water.

### DETAILED DESCRIPTION

A water-dispersible wet wipe includes a fibrous material, a binder composition containing an ionic copolymer, and an aqueous wetting composition containing a salt and at least about 10 weight percent of an organic solvent. The wet wipe is not dispersible in the wetting composition, but is dispersible in hard or soft water. The mixed solvent system of the wetting composition may provide cleaning properties that are not feasible with aqueous compositions containing less than 10 weight percent organic solvent. Accordingly, the water-dispersible wet wipes may be useful for household cleansing, hard surface cleaning, medical wipes and industrial cleaning, in addition to personal cleaning such as treating skin, make-up removal and nail polish removal. The dispersibility properties of the wet wipe may provide for mechanical strength during use, while also being flushable to allow for ease of disposal.

Ionic Copolymers

Binder compositions for water-dispersible wet wipes contain an ionic copolymer. As used herein, the term “ionic copolymer” refers to a polymer having at least two different species of monomeric units, where one of the species is neutral, and one species contains an ionic group. The ionic group may be either cationic or anionic. An ionic copolymer may include more than one species of ionic monomeric units and/or more than one species of neutral monomeric units. If two or more species of ionic monomeric units are present, the charge on these units may be the same, or they may be different. If two or more species of neutral monomeric units are present, these units may independently be hydrophilic or hydrophobic. The binder composition may also include a co-binder polymer, which may be a neutral polymer or may contain at least one ionic group.

Ionic copolymers may be used, alone or in combination with other materials, as binder compositions for fibrous materials. Binder compositions containing an ionic copolymer may be non-dispersible, or they may be water-dispersible, providing for flushability of products containing the binder compositions. In order to be effective for flushable products, a binder composition should disperse in water from any available water supply, such as tap water, whether that water is “soft water” or “hard water.” Based on a study conducted by the American Chemical Society, water hardness across the United States varies greatly, with CaCO₃ concentration ranging from near zero for soft water to about 500 ppm CaCO₃ (about 200 ppm Ca²⁺ ion) for very hard water. The same binder composition should remain stable and maintain product integrity under dry environments or under wet environments having relatively high concentrations of monovalent and/or divalent ions.

Ionic copolymers may contribute to the water-dispersibility of a binder composition by having aqueous solubility properties that vary depending on the type and/or amount of ions present in the aqueous environment. First, an ionic copolymer preferably is soluble in water containing up to about 200 ppm or more divalent ions, particularly calcium or magnesium. Second, an ionic copolymer preferably is insoluble in a salt solution containing at least about 0.3 weight percent of one or more inorganic and/or organic salts containing monovalent and/or divalent ions.

Desirably, an ionic copolymer is soluble in water containing up to about 50 ppm Ca²⁺ and/or Mg²⁺ ions. More desirably, an ionic copolymer is soluble in water containing up to about 100 ppm Ca²⁺ and/or Mg²⁺ ions. Even more desirably, an ionic copolymer is soluble in water containing up to about 150 ppm Ca²⁺ and/or Mg²⁺ ions. Even more desirably, an ionic copolymer is soluble in water containing up to about 200 ppm Ca²⁺ and/or Mg²⁺ ions.

Desirably, an ionic copolymer is insoluble in a salt solution containing from about 0.3% to about 10% by weight of one or more inorganic and/or organic salts containing monovalent and/or divalent ions. More desirably, an ionic copolymer is insoluble in salt solutions containing from about 0.5% to about 5% by weight of one or more inorganic and/or organic salts containing monovalent and/or divalent ions. Even more desirably, an ionic copolymer is insoluble in salt solutions containing from about 1.0% to about 4.0% by weight of one or more inorganic and/or organic salts containing monovalent and/or divalent ions. Examples of monovalent ions include, but are not limited to, Na⁺ ions, K⁺ ions, Li⁺ ions, NH₄₊ ions, low molecular weight quaternary ammonium compounds (e.g., those having fewer than 5 carbons on any side group), and a combination thereof. Examples of multivalent ions include, but are not limited to, Zn²⁺, Ca²⁺ and Mg²⁺. The monovalent and divalent ions may be derived from organic and inorganic salts including, but not limited to, NaCl, NaBr, KCl, NH₄Cl, Na₂SO₄, ZnCl₂, CaCl₂, MgCl₂, MgSO₄, NaNO₃, NaSO₄CH₃, and combinations thereof. Typically, alkali metal halides are most desirable because of cost, purity, low toxicity, and availability. A particularly desirable salt is NaCl.

A wet wipe may be prepared by treating a fibrous web with a binder composition, optionally drying the binder composition, then contacting the treated fibrous web with a wetting composition. A controlled concentration of salt in the wetting composition can insolubilize the binder composition and allow it to function as an adhesive for the fibrous material. However, when the wet wipe is diluted with water, as occurs when it is discarded into a wastewater stream, the salt concentration is diluted. As a result, the ionic copolymer becomes soluble, the strength of the wet wipe drops below a critical level, and the wet wipe will break apart into small pieces and disperse. Thus the water-dispersible wet wipes maintain integrity or wet strength during storage and use, but break apart or disperse after disposal in the toilet, when the salt or ion concentration falls below the critical level.

A cationic copolymer may be used as the ionic copolymer in the binder composition. In addition to one or more cationic monomeric units, the cationic copolymer contains one or more non-ionic monomeric units. The non-ionic monomeric units may be hydrophobic or hydrophilic, or may contain a mixture of hydrophilic and hydrophobic units. Examples of cationic copolymers that are soluble in hard or soft water are disclosed in U.S. Patent Application Publication Nos. 2003/0026963 A1; 2003/0027470 A1; 2003/0032352 A1; 2004/0030080 A1; 2003/0055146 A1; 2003/0022568 A1; 2003/0045645 A1; 2004/0058600 A1; 2004/0058073 A1; 2004/0063888 A1; 2004/0055704 A1; 2004/0058606 A1; and 2004/0062791 A1; all of which are incorporated herein by reference.

An example of a cationic copolymer is represented by the following formula: 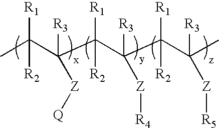
wherein x=1 to about 15 mole percent; y=about 60 to about 99 mole percent; z=0 to about 30 mole percent; Q is C₁-C₄ alkyl ammonium, quaternary C₁-C₄ alkyl ammonium or benzyl ammonium; Z is —O—, —C(═O)O—, —O(O═)C—, —C(═O)NH—, and —NHC(═O)—; R₁, R₂, R₃ are independently hydrogen or methyl; R₄ is methyl and ethyl; and R₅ is an alkyl group such as methyl, ethyl, butyl, ethylhexyl, decyl or dodecyl, or a heteroalkyl group such as hydroxyethyl, hydroxypropyl, polyoxyethylene, or polyoxypropylene.

A variety of monomers can be used to provide the cationic monomeric units, represented as Q-containing monomeric units in the above formula. Examples of cationic monomers include [2-(acryloxy)ethyl]trimethyl ammonium chloride (ADAMQUAT), [2-(methacryloxy)ethyl)trimethyl ammonium chloride (MADQUAT), (3-acrylamidopropyl) trimethyl ammonium chloride, N,N-diallyldimethyl ammonium chloride, [2-(acryloxy) ethyl]dimethylbenzyl ammonium chloride, (2-(methacryloxy) ethyl]dimethylbenzyl ammonium chloride, [2-(acryloxy)ethyl]dimethyl ammonium chloride, and [2-(methacryloxy)ethyl]dimethyl ammonium chloride. Cationic monomeric units can also be provided by precursor monomers, such as vinylpyridine, dimethylaminoethyl acrylate, and dimethylaminoethyl methacrylate, which can be polymerized and then quaternized through post-polymerization reactions, such as by treatment with a quaternization reagent. Monomers or quaternization reagents which provide different counter-ions, such as bromide, iodide, or methyl sulfate are also useful.

A variety of monomers can be used to provide hydrophobic monomeric units, represented as the R₄- and R₅-containing monomeric units in the above formula. Examples of hydrophobic monomers include, but are not limited to, branched or linear C₁-C₁₈ alkyl vinyl ethers, vinyl esters, acrylamides, acrylates, and other monomers that can be copolymerized with the cationic monomer. As used herein methyl acrylate is considered to be a hydrophobic monomer. Methyl acrylate has a solubility of 6 g/100 ml in water at 20° C.

Another example of a cationic copolymer is represented by the following formula: 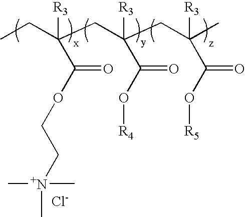
where x, y, z, R₄ and R₅ are as defined above.

Yet another example of a cationic copolymer is represented by the following formula: 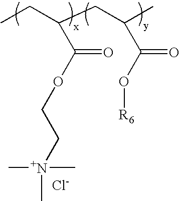
where x=1 to about 15 mole percent; y=about 85 to about 99 mole percent and R₆ is C₁-C₄ alkyl. In a specific example of a copolymer having this formula, R₆ is methyl, x=3 to about 6 mole percent; and y=about 94 to about 97 mole percent.

Desirably, these cationic copolymers are insoluble in a salt solution containing at least about 0.3 weight percent of one or more inorganic and/or organic salts containing monovalent and/or divalent ions. More desirably, these cationic copolymers are insoluble in a salt solution containing from about 0.3% to about 10% by weight of one or more of these salts. More desirably, these cationic copolymers are insoluble in a salt solution containing from about 0.5% to about 5% by weight of one or more of these salts, or from about 1.0% to about 4.0% by weight of one or more of these salts. Examples of monovalent ions include, but are not limited to, Na⁺ ions, K⁺ ions, Li⁺ ions, NH₄₊ ions, low molecular weight quaternary ammonium compounds (e.g., those having fewer than 5 carbons on any side group), and a combination thereof. Suitable multivalent ions include, but are not limited to, Zn²⁺, Ca²⁺ and Mg²⁺. The monovalent and divalent ions can be derived from organic and inorganic salts including, but not limited to, NaCl, NaBr, KCl, NH₄Cl, Na₂SO₄, ZnCl₂, CaCl₂, MgCl₂, MgSO₄, NaNO₃, NaSO₄CH₃, and combinations thereof. Typically, alkali metal halides are most desirable because of cost, purity, low toxicity, and availability. A particularly desirable salt is NaCl.

Another example of a cationic copolymer includes copolymers formed from two, three or four different monomers, where the monomers include a cationic monomer and at least one hydrophobic monomer. These copolymers may be terpolymers or tetrapolymers that are the polymerization products of a cationic monomer, at least one hydrophobic monomer, and optionally at least one hydrophilic monomer. The cationic monomer may be any of the cationic monomers listed above. The hydrophobic monomers may be any of the classes of hydrophobic monomers listed above. Specifically, the hydrophobic monomers may be alkyl acrylates, such as butyl acrylate, 2-ethylhexyl acrylate, ethyl acrylate, lauryl acrylate, and hexadecyl acrylate; methacrylate analogs of these alkyl acrylates; and combinations of these monomers. Examples of hydrophilic monomers include, but are not limited to, acrylamide and methacrylamide based monomers, such as acrylamide, N,N-dimethyl acrylamide, N-ethyl acrylamide, N-isopropyl acrylamide and hydroxymethyl acrylamide; hydroxyalkyl acrylates and hydroxyalkyl methacrylates, such as hydroxyethyl methacrylate and hydroxyethyl acrylate; polyalkoxyl acrylates and polyalkoxyl methacrylates, such as polyethyleneglycol acrylates and polyethyleneglycol methacrylates (“PEG-MA”); N-vinylpyrrolidinone; and N-vinylformamide.

In a specific example of these cationic copolymers, the cationic copolymer is the polymerization product of the following four monomers: acrylamide, butyl acrylate, 2-ethylhexyl acrylate and [2-(methacryloyloxy)ethyl]trimethyl ammonium chloride. In another specific example, the cationic copolymer is formed from three different monomers: butyl acrylate, 2-ethylhexyl acrylate and [2-(methacryloyloxy)ethyl]trimethyl ammonium chloride. In another specific example, the cationic copolymer is the polymerization product of [2-(methacryloyloxy)ethyl]trimethyl ammonium chloride and butyl acrylate or 2-ethylhexyl acrylate. In another specific example, the cationic copolymer is the polymerization product of [2-(methacryloyloxy)ethyl]trimethyl ammonium chloride, butyl acrylate and 2-ethylhexyl acrylate.

Desirably, cationic copolymers formed from 2, 3 or 4 different monomers, where the monomers include a cationic monomer and at least one hydrophobic monomer, are insoluble in a salt solution containing at least about 2 weight percent of one or more inorganic and/or organic salts containing monovalent and/or multivalent ions. More desirably, these cationic copolymers are insoluble in a salt solution containing from about 2 weight percent to about 5 weight percent of one or more inorganic and/or organic salts containing monovalent and/or multivalent ions. Even more desirably, these cationic copolymers are insoluble in salt solutions containing from about 2 weight percent to about 4 weight percent of one or more inorganic and/or organic salts containing monovalent and/or multivalent ions. Examples of monovalent ions for these copolymers include, but are not limited to, Na⁺ ions, K⁺ ions, Li⁺ ions, NH₄₊ ions, low molecular weight quaternary ammonium compounds (e.g., those having fewer than 5 carbons on any side group), and a combination thereof. Examples of multivalent ions for these copolymers include, but are not limited to, Zn²⁺ and Ca²⁺.

An anionic copolymer may be used as the ionic copolymer in the binder composition. In addition to one or more anionic monomeric units, the anionic copolymer contains one or more non-ionic monomeric units. The non-ionic monomeric units may be hydrophobic or hydrophilic, or may contain a mixture of hydrophilic and hydrophobic units. Examples of anionic copolymers that are soluble in hard or soft water are disclosed in U.S. Pat. Nos. 6,683,143 B1; 6,602,955 B2; 6,815,502 B1; 6,599,848 B1; 6,814,974 B1; 6,713,414 B1; and 6,653,406 B1; all of which are incorporated herein by reference.

An example of an anionic copolymer is a sulfonate anion modified acrylic acid copolymer. In one example, sulfonate anion modified acrylic acid copolymers may be formed by copolymerizing at least one sulfonate-containing monomer, at least one vinyl carboxylate monomer, and at least one non-ionic monomer. Examples of sulfonate-containing monomers include, but are not limited to, 2-acrylamido-2-methyl-1-propanesulfonic acid (AMPS) and organic or inorganic salts of 2-acrylamido-2-methyl-1-propanesulfonic acid, such as alkali earth metal and organic amine salts of 2-acrylamido-2-methyl-1-propanesulfonic acid, particularly the sodium salt of 2-acrylamido-2-methyl-1-propanesulfonic acid (NaAMPS). Additional sulfonate-containing monomers include, but are not limited to, 2-methyl-2-propene sulfonic acid, vinyl sulfonic acid, styrene sulfonic acid, 2-sulfopropyl methacrylate and 3-sulfopropyl acrylate, and organic or inorganic salts thereof, such as alkali earth metals and organic amine salts, such as alkyl ammonium hydroxide wherein the alkyl groups are C₁-C₁₈. Examples of vinyl carboxylate monomers include, but are not limited to, acrylic acid, methacrylic acid, and itaconic acid.

A variety of non-ionic monomers can be used to form the non-ionic monomeric units in an anionic copolymer. Examples of non-ionic monomers include, but are not limited to, acrylic esters and methacrylic esters having an alkyl group of 1 to 18 carbon atoms or having a cycloalkyl group of 3 to 18 carbon atoms. Preferably acrylic esters and methacrylic esters used as non-ionic monomer have an alkyl group of 1 to 12 carbon atoms or a cycloalkyl group of 3 to 12 carbon atoms. Other examples of non-ionic monomers include, but are not limited to, acrylamide and methacrylamide based monomers, such as acrylamide, N,N-dimethyl acrylamide, N-ethyl acrylamide, N-isopropyl acrylamide, and hydroxymethyl acrylamide; N-vinylpyrrolidinone; N-vinylformamide; hydroxyalkyl acrylates and hydroxyalkyl methacrylates, such as hydroxyethyl methacrylate and hydroxyethyl acrylate.

The relative amounts monomers that may be employed in the formation of the anionic copolymer may vary depending on the desired properties in the resulting anionic copolymer. The mole percent of acrylic acid monomer that may be utilized for formation of the anionic copolymer may be up to about 70 mole percent. An anionic copolymer may be produced from a mixture of monomers containing 10 weight percent (wt %) to 90 wt %, desirably 20 wt % to 70 wt % of acrylic acid and/or methacrylic acid, and 90 wt % to 10 wt %, desirably 80 wt % to 30 wt % of acrylic esters and/or methacrylic esters having an alkyl group of 1 to 18 carbon atoms or a cycloalkyl group of 3 to 18 carbon atoms in which 1 to 60 mole percent, desirably 5 to 50 mole percent of acrylic acid and/or methacrylic acid is neutralized to form a salt; or copolymers of 30 wt % to 75 wt %, desirably 40 wt % to 65 wt % of acrylic acid, 5 wt % to 30 wt %, desirably 10 wt % to 25 wt % of acrylic esters and/or methacrylic esters having an alkyl group of 8 to 12 carbon atoms and 20 wt % to 40 wt %; desirably 25 wt % to 35 wt % of acrylic esters and/or methacrylic esters having an alkyl group of 2 to 4 carbon atoms in which 1 to 50 mole percent, desirably 2 to 40 mole percent of acrylic acid is neutralized to form a salt.

In one example, an anionic copolymer may be produced from the following monomers: acrylic acid, methacrylic acid, or a combination thereof; 2-acrylamido-2-methyl-1-propanesulfonic acid (AMPS) and organic or inorganic salts thereof, such as the sodium salt thereof (NaAMPS); butyl acrylate; and 2-ethylhexyl acrylate. In a preferred example, the anionic co-polymer may be produced from: acrylic acid; AMPS, NaAMPS or a combination thereof; butyl acrylate; and 2-ethylhexyl acrylate. In another example, an anionic copolymer may also be produced by sulfonation of an existing polymer, such as an acrylic acid-derived terpolymer. Methods for the production of sulfonated or sulfated polymers are disclosed in U.S. Pat. Nos. 3,624,069; 4,419,403; 5,522,967; 4,220,739; 5,783,200; 2,400,720; 2,937,066; 2,786,780; 2,832,696; 3,613,957, and 3,740,258, all of which are herein incorporated by reference.

Ionic copolymers may be prepared according to a variety of polymerization methods. A solution polymerization method is desired, as this method can provide for a random distribution of monomer units. Suitable solvents for the polymerization method include, but are not limited to, lower alcohols, such as methanol, ethanol and propanol; a mixed solvent of water and one or more lower alcohols mentioned above; and a mixed solvent of water and one or more lower ketones, such as acetone or methyl ethyl ketone.

Any free radical polymerization initiator may be used in the polymerization. Selection of a particular initiator may depend on a number of factors including, but not limited to, the polymerization temperature, the solvent, and the monomers used. Suitable polymerization initiators include, but are not limited to, 2,2′-azobisisobutyronitrile, 2,2′-azobis(2-methylbutyronitrile), 2,2′-azobis(2,4-dimethylvaleronitrile), 2,2′-azobis(2-amidinopropane)-dihydrochloride, 2,2′-azobis(N,N′-dimethyleneisobutylamidine), potassium persulfate, ammonium persulfate, and aqueous hydrogen peroxide. The amount of polymerization initiator may desirably range from about 0.01 to 5 weight percent based on the total weight of monomer present. The polymerization temperature may vary depending on the polymerization solvent, monomers, and initiator used, but in general, ranges from about 20° C. to about 90° C. Polymerization time generally ranges from about 2 to about 8 hours. Ionic copolymers may have an average molecular weight that varies depending on the ultimate use of the polymer. The ionic copolymer may have a weight average molecular weight ranging from about 10,000 to about 5,000,000 grams per mol. More specifically, the ionic copolymer have a weight average molecular weight ranging from about 25,000 to about 2,000,000 grams per mol., or, more specifically still, from about 200,000 to about 1,000,000 grams per mol.

Binder Compositions And Fibrous Substrates Containing The Compositions

In addition to providing for wet strength in the presence of the wetting composition while also providing for dispersibility in hard or soft water, the binder composition may have other desirable properties. For example, it is preferred that the binder composition is processable on a commercial basis; i.e., may be applied relatively quickly on a large scale basis, such as by spraying, which thereby requires that the binder composition have a relatively low viscosity at high shear. It is also preferred that the binder composition provide acceptable levels of sheet wettability with the wetting composition, of sheet stiffness, and of sheet tackiness. In addition, it is preferred that all components of the wet wipe, including the binder composition, are non-toxic and relatively economical.

Binder compositions for water-dispersible wet wipes may also include a co-binder polymer, in addition to the ionic copolymer. A co-binder polymer in the binder composition can reduce the shear viscosity of the ionic copolymer, such that a binder composition containing the ionic copolymer and the co-binder polymer is sprayable. Sprayability of the binder composition allows the composition to be applied to a web of fibrous material in a uniform manner, including uniform distribution of the composition across the material uniform penetration of the composition into the material. A co-binder polymer may also provide for a reduction in the stiffness of a fibrous substrate formed by applying the binder composition to a fibrous material. In one example, a co-binder polymer can be substituted for a portion of the ionic copolymer in a binder composition without impairing the strength of fibrous substrates containing the binder composition. This substitution may improve properties of the fibrous substrate, such as by lowering the substrate stiffness, by improving tactile properties of the substrate such as lubricity and smoothness, and/or by reducing the cost of the substrate.

Preferably the co-binder polymer is largely dispersed in the ionic copolymer, such that the ionic copolymer is the continuous phase and the co-binder polymer is the discontinuous phase. Desirably, the co-binder polymer can also meet several additional criteria. For example, it may be desirable for the co-binder polymer to have a glass transition temperature that is lower than the glass transition temperature of the ionic copolymer. The co-binder may be present at a level relative to the solids mass of the ionic copolymer of about 45% or less, preferably about 30% or less, more preferably about 20% or less, more preferably about 15% or less, and more preferably about 10% or less, with exemplary ranges of from about 1% to about 45% or from about 25% to about 35%, as well as from about 1% to about 20% or from about 5% to about 25%. The amount of co-binder present should be low enough, that the co-binder remains a discontinuous phase unable to create enough crosslinked, or insoluble bonds, to jeopardize the dispersibility of the treated substrate.

Examples of co-binder polymers and their use in binder compositions are given in the above mentioned U.S. Patent Application Publication Nos. 2003/0026963 A1; 2003/0027470 A1; 2003/0032352 A1; 2004/0030080 A1; 2003/0055146 A1; 2003/0022568 A1; 2003/0045645 A1; 2004/0058600 A1; 2004/0058073 A1; 2004/0063888 A1; 2004/0055704 A1; 2004/0058606 A1; and 2004/0062791 A1 and U.S. Pat. Nos. 6,683,143 B1; 6,602,955 B2; 6,815,502 B1; 6,599,848 B1; 6,814,974 B1; 6,713,414 B1; and 6,653,406 B1. Specific examples of co-binder polymers include latex emulsions, which may be weakly anionic, nonionic, or cationic. A specific example of a latex co-binder polymer is poly(ethylene-vinyl acetate), such as Dur-O-Set® RB available from National Starch and Chemical Co., Bridgewater, N.J. In one example, a binder composition includes about 55 to about 95 weight percent ionic copolymer and about 5 to about 45 weight percent poly(ethylene-vinyl acetate). In another example, a binder composition includes about 75 weight percent ionic copolymer and about 25 weight percent poly(ethylene-vinyl acetate).

A binder composition, with or without a co-binder polymer, may be applied to any fibrous material including, but are not limited to, nonwoven and woven fabrics. In many personal care products, preferred fibrous materials are nonwoven fabrics. As used herein, the term “nonwoven fabric” refers to a sheet that has a structure of individual fibers or filaments randomly arranged in a mat-like fashion (including papers). Nonwoven fabrics can be made from a variety of processes including, but not limited to, air-laid processes, wet-laid processes, hydroentangling processes, staple fiber carding and bonding, and solution spinning.

The binder composition may be applied to the fibrous material by any known process of application. Suitable processes for applying the binder composition include, but are not limited to, printing, spraying, electrostatic spraying, coating, flooded nips, metered press rolls, impregnating or by any other technique. The amount of binder composition may be metered and distributed uniformly within the fibrous material or may be non-uniformly distributed within the fibrous material. The binder composition may be distributed throughout the entire fibrous material or it may be distributed within a multiplicity of small closely spaced areas. Uniform distribution of binder composition is typically desired.

For ease of application to the fibrous material, the binder composition may be dissolved in water, or in a non-aqueous solvent, such as methanol, ethanol, acetone, or the like, with water being the preferred solvent. The amount of binder composition dissolved in the solvent may vary, depending on the polymer used and the substrate application. Desirably, the binder solution contains up to about 50 percent by weight of binder composition solids. More desirably, the binder solution contains from about 10 to 30 percent by weight of binder composition solids, especially about 15-25 percent by weight binder composition solids. Plasticizers, perfumes, coloring agents, antifoams, bactericides, preservative, surface active agents, thickening agents, fillers, opacifiers, tackifiers, detackifiers, and similar additives can be incorporated into the solution of binder components, if so desired.

Once a binder composition is applied to the fibrous material, the mixture is dried by any conventional means. Once dry, the resultant fibrous substrate exhibits improved tensile strength when compared to the tensile strength of the untreated wet-laid or dry-laid fibrous materials, and yet has the ability to rapidly “fall apart”, or disintegrate when placed in soft or hard water having a divalent ion concentration up to about 200 ppm and agitated. For example, the dry tensile strength of the fibrous substrate may be increased by at least 25 percent as compared to the dry tensile strength of the untreated fibrous material not containing the binder composition. More particularly, the dry tensile strength of the fibrous substrate may be increase by at least 100 percent as compared to the dry tensile strength of the untreated fibrous material not containing the binder composition. Even more particularly, the dry tensile strength of the fibrous substrate may be increased by at least 500 percent as compared to the dry tensile strength of the untreated fibrous material not containing the binder composition.

A desirable feature of binder compositions containing ionic copolymers is that the improvement in tensile strength may be effected where the amount of binder composition present (“add-on”) in the resultant fibrous substrate represents only a small portion by weight of the entire substrate. “Add-on” is equal to the mass of binder composition divided by the mass of dry fiber. Generally add-on is expressed as a percentage. The amount of add-on can vary for a particular application; however, the optimum amount of add-on results in a fibrous substrate which has integrity and is not dispersible while in use and also quickly disperses when soaked in water. For example, the binder composition typically is from about 5 to about 65 percent, by weight, of the total weight of the substrate. More particularly, the binder composition may be from about 7 to about 35 percent, by weight, of the total weight of the substrate. Even more particularly, the binder composition may be from about 10 to about 20 percent by weight of the total weight of the substrate.

Fibrous substrates having a binder composition containing an ionic copolymer desirably have good in-use tensile strength, as well as water-dispersibility. Desirably, these fibrous substrates are abrasion resistant and retain significant tensile strength in aqueous solutions containing the specific amount and type of ions disclosed above. Because of this latter property, the fibrous substrates are well suited for disposable products, such as sanitary napkins, diapers, adult incontinence products, and dry and wet wipes, which may be disposed of in a flush toilet in any part of the world.

The fibers forming the fibrous substrates can be made from a variety of materials including natural fibers, synthetic fibers, and combinations thereof. The choice of fibers depends upon, for example, the intended end use of the finished substrate and fiber cost. For instance, suitable fibrous substrates may include, but are not limited to, natural fibers such as cotton, linen, jute, hemp, wool, wood pulp, etc. Similarly, regenerated cellulosic fibers, such as viscose rayon and cuprammonium rayon, modified cellulosic fibers, such as cellulose acetate, or synthetic fibers, such as those derived from polypropylenes, polyethylenes, polyolefins, polyesters, polyamides, polyacrylics, etc., alone or in combination with one another, may likewise be used. Blends of one or more of the above fibers may also be used, if so desired. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Mercerized, chemically stiffened or crosslinked fibers may also be used.

Synthetic cellulose fiber types include rayon in all its varieties and other fibers derived from viscose or chemically modified cellulose, including regenerated cellulose and solvent-spun cellulose, such as Lyocell. Chemically treated natural cellulosic fibers can be used, such as mercerized pulps, chemically stiffened or crosslinked fibers, or sulfonated fibers. Recycled fibers, as well as virgin fibers, can be used. Cellulose produced by microbes and other cellulosic derivatives can be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. A binder composition may also be applied to other fibers or particles. Other fibers that may be treated with the binder composition include fibers such as those made from carboxymethyl cellulose, chitin, and chitosan. The binder composition may also be applied to particles, such as sodium polyacrylate superabsorbent particles. Superabsorbent particles are frequently incorporated on or into fibrous substrates used for personal care items, especially nonwoven fabrics.

The fiber length can affect the properties of the fibrous substrate. In some examples, such as flushable products, fiber length is of more importance. The minimum length of the fibers depends on the method selected for forming the fibrous substrate. For example, where the fibrous substrate is formed by carding, the length of the fiber should usually be at least about 42 mm in order to insure uniformity. Where the fibrous substrate is formed by air-laid or wet-laid processes, the fiber length may desirably be about 0.2 to 6 mm. Although fibers having a length of greater than 50 mm can be used, it has been determined that when a substantial quantity of fibers having a length greater than about 15 mm is placed in a flushable fibrous substrate, though the fibers will disperse and separate in water, their length tends to form “ropes” of fibers, which are undesirable when flushing in home toilets. Therefore, for these products, it is desired that the fiber length be about 15 mm or less so that the fibers will not have a tendency to “rope” when they are flushed through a toilet. Although fibers of various lengths are applicable, desirably fibers are of a length less than about 15 mm so that the fibers disperse easily from one another when in contact with water. The fibers, particularly synthetic fibers, can also be crimped.

Fibrous substrates containing a binder composition may be formed from a single layer or multiple layers. In the case of multiple layers, the layers are generally positioned in a juxtaposed or surface-to-surface relationship and all or a portion of the layers may be bound to adjacent layers. Fibrous substrates may also be formed from a plurality of separate fibrous substrates wherein the separate fibrous substrates may be formed from single or multiple layers. In those instances where the fibrous substrate includes multiple layers, the entire thickness of the fibrous substrate may be subjected to a binder composition application, or each individual layer may be separately subjected to a binder composition application and then combined with other layers in a juxtaposed relationship to form the finished fibrous substrate.

Binder compositions containing an ionic copolymer are particularly useful for binding fibers of air-laid nonwoven fabrics. These air-laid materials are useful for body-side liners, fluid distribution materials, fluid in-take materials, such as a surge material, absorbent wrap sheet and cover stock for various water-dispersible personal care products. Air-laid materials are particularly useful for use as a wet wipe (pre-moistened wipe). The basis weights for air-laid non-woven fabrics may range from about 20 to about 200 grams per square meter (“gsm”) with staple fibers having a denier of about 0.5-10 and a length of about 6-15 millimeters. Surge, or in-take, materials need better resiliency and higher loft so staple fibers having about 6 denier or greater are used to make these products. A desirable final density for the surge, or in-take, materials is between about 0.025 grams per cubic centimeter (“g/cc”) to about 0.10 g/cc. Fluid distribution materials may have a higher density, in the desired range of about 0.10 to about 0.20 g/cc using fibers of lower denier, most desirable fibers have a denier of less than about 1.5. Wipes generally can have a fiber density of about 0.025 g/cc to about 0.2 g/cc and a basis weight of about 20 gsm to about 150 gsm; specifically from about 30 to about 90 gsm, and most specifically from about 60 gsm to about 65 gsm.

The in-use tensile strength of a fibrous substrate may be enhanced by forming the fibrous substrate with a binder composition containing an ionic copolymer and subsequently applying either one or more monovalent and/or divalent salts to the fibrous substrate. The salt may be applied to the fibrous substrate by any method known to those of ordinary skill in the art including, but not limited to, applying a solid powder onto the substrate and spraying a salt solution onto the substrate. The amount of salt may vary depending on a particular application. However, the amount of salt applied to the substrate is typically from about 0.3 weight percent to about 10 weight percent salt solids based on the total weight of the substrate.

Unlike other binder systems, compositions containing an ionic copolymer may be activated as binders without the need for elevated temperature. While drying or water removal is useful in achieving a good distribution of the binder composition in a fibrous substrate, elevated temperature, per se, is not essential because the binder composition does not require crosslinking or other chemical reactions with high activation energy to serve as a binder. Rather, the interaction with a soluble insolubilizing compound, typically a salt, is sufficient to cause the binder composition to become insoluble; i.e., “salted out” or activated by interaction between the salt and the cation and/or anion attached to the polymer. Thus, a drying step can be avoided, if desired, or replaced with low-temperature water removal operations such as room-temperature drying or freeze drying. Elevated temperature is generally helpful for drying, but the drying can be done at temperatures below what is normally needed to drive crosslinking reactions. Thus, the peak temperature to which the substrate is exposed or to which the substrate is brought can be below any of the following: 200° C., 180° C., 160° C., 140° C., 120° C., 110° C., 105° C., 100° C., 90° C., 75° C., and polymer systems, such as commercial latex emulsions, may also comprise crosslinkers suited for reaction at temperatures of 160° C. or higher, maintaining a lower peak temperature can be beneficial in preventing development of excessive strength in the polymer that might otherwise hinder the water dispersibility of the wet wipe.

Water-Dispersible Wet Wipes

Fibrous substrates having a binder composition containing an ionic copolymer can be used as wet wipes, when wetted with an appropriate wetting composition. For wet wipes that can be used for surface cleaning, the wetting composition preferably contains water, a salt, and at least about 10 wt % organic solvent. Examples of organic solvents include, but are not limited to, alcohols, ketones, ethers, acetates, amines, amides, sulfides, sulfoxides, and halogenated hydrocarbons. Specific examples of organic solvents that may be present in the wetting composition include alcohols such as methanol, ethanol, iso-propanol, n-propanol, butyl alcohol, and glycerol; ketones such as acetone, methyl ethyl ketone (MEK) and methyl isobutyl ketone (MIBK); ethers such as diethyl ether and tetrahydrofuran (THF); acetates such as methyl acetate and ethyl acetate; amides such as dimethylformamide (DMF) and N-methyl-2-pyrrolidone (NMP); sulfides such as carbon disulfide; sulfoxides such as dimethyl sulfoxide (DMSO); and halogenated hydrocarbons such as methylene chloride.

Desirably, the wetting composition contains more than about 10 weight percent of an organic solvent based on the total weight of the wetting composition. Preferably, the wetting composition may contain from about 10 weight percent to about 75 weight percent of an organic solvent. More preferably, the wetting composition may contain from about 10 weight percent to about 50 weight percent of an organic solvent. More preferably, the wetting composition may contain from about 10 weight percent to about 30 weight percent of an organic solvent.

The organic solvent may be miscible with water to provide a solution, or it may be immiscible with water to provide an emulsion. Preferably the organic solvent is miscible with water. More preferably the organic solvent is a lower alcohol or a ketone. In one example, the organic solvent is an alcohol. Ethanol and isopropanol have long histories of safe use for skin-contact applications, and may also impart antimicrobial properties to the wet wipe. Glycerol also has a long history of safe use for skin-contact applications, and may serve as an emollient in a wet wipe solution. In another example, the organic solvent is a ketone such as acetone, which is commonly used for removal of nail polish.

Water-dispersible wet wipes containing an organic solvent in the wetting composition can provide for enhanced properties relative to conventional wet wipes. The properties include, for example, cleaning of household and institutional surfaces, antiseptic wiping, and cleaning of medical waste from surfaces or from human or animal organisms. The water-dispersibility of the wipes allows for a used wipe, including the soil on the surface of the used wipe, to be easily disposed of by flushing down the drain. In one example, a water-dispersible wet wipe may be used to remove stains that are impervious to conventional aqueous wetting compositions, such as ink and grease. The presence of solvents such as iso-propanol or acetone in the wetting composition may provide for removal of these substances.

In another example, a wet wipe containing an alcohol and a salt may provide for a synergistic effect in antimicrobial wiping. It has been reported that ethanol and NaCl together can produce an antimicrobial effect at lower concentrations than when either ingredient is present alone. The presence of ethanol and NaCl may also enhance the efficacy of antimicrobial additives in the wet wipe. See, for example, J D Campo, M Amiot, C Nguyen-The, “Antimicrobial Effect of Rosemary Extracts,” Journal of Food Protection, 63 (10), 1359-1368, 2000; and N Kurita, S Koike, “Synergistic Antimicrobial Effect of Ethanol, Sodium Chloride, Acetic Acid and Essential Oil Components,” Agric. Biol. Chem., 47(1), 67-75, 1983.

In another example, a wet wipe containing an alcohol may be used in a variety of medical procedures, such as cleansing the skin of a human or animal patient prior to giving an injection. After use, conventional wipes or swabs may be considered “medical waste,” the proper disposal of which can be expensive and burdensome. Since ordinary toilet and municipal sewer systems are intended to safely dispose of biological waste, the use of a water-dispersible wet wipe containing an alcohol in the wetting composition may provide an acceptable alternative to disposal as medical waste.

In another example, a wet wipe containing an organic solvent may be used to clean and dry a surface. Alcohols in particular are capable of forming minimum boiling azeotropes with water. Thus, a wetting composition may be formulated such that the azeotrope removes water as it evaporates, providing for drying of the surface. This property may be particularly advantageous, for example, in cleaning a toilet seat. In skin care applications the rapid evaporation of the wetting composition may provide comfort for warm, irritated, or itchy skin. Table 1 below lists some common alcohol—water azeotropes, taken from Lang”s Handbook of Chemistry. Ideally, the azeotrope combines a low boiling point alcohol and a high content of water in the azeotrope.

TABLE 1
Alcohol-Water Azeotropes

| | boiling point of | % water in |
|---|---|---|
| Alcohol | azeotrope (° C.) | azeotrope |
| Ethyl | 78:1 | 4.5 |
| iso-propyl | 80.4 | 12.1 |
| n-propyl | 87.7 | 28.3 |
| sec-butyl | 88.5 | 32.1 |
| iso-butyl | 90.0 | 33.2 |
| n-butyl | 92.4 | 38.0 |

The fibrous material may be in the form of a woven or nonwoven fabric; however, nonwoven fabrics are more desirable. The nonwoven fabric is desirably formed from relatively short fibers, such as wood pulp fibers. The minimum length of the fibers depends on the method selected for forming the nonwoven fabric. Where the nonwoven fabric is formed by a wet or dry method, the fiber length is desirably from about 0.1 millimeters to 15 millimeters. Desirably, the nonwoven fabric has a relatively low wet cohesive strength when it is not bonded together by an adhesive or binder composition. When such nonwoven fabrics are bonded together by a binder composition, which loses its bonding strength in tap water and in sewer water, the resulting fibrous substrate will break up readily by the agitation provided by flushing and moving through the sewer pipes.

The finished wipes may be individually packaged, desirably in a folded condition, in a moisture proof envelope or packaged in containers holding any desired number of sheets in a water-tight package with a wetting composition applied to the wipe. The finished wipes may also be packaged as a roll of separable sheets in a moisture-proof container holding any desired number of sheets on the roll with a wetting composition applied to the wipes. The roll can be coreless and either hollow or solid. Coreless rolls, including rolls with a hollow center or without a solid center, can be produced with known coreless roll winders, including those of SRP Industry, Inc. (San Jose, Calif.); Shimizu Manufacturing (Japan), and the devices disclosed in U.S. Pat. No. 4,667,890, issued May 26, 1987 to Gietman. Solid-wound coreless rolls can offer more product for a given volume and can be adapted for a wide variety of dispensers.

Wipes may also be prepared by applying a binder composition to a fibrous material, drying the resulting fibrous substrate, and then packaging the dry substrate as a wipe. In this example, a wetting composition is added some time later. For example, large rolls or stacks of dry wipers are prepared as an intermediate material, prior to treatment with the wetting solution and subsequent packaging of the wet wipes. This procedure may be advantageous as part of the manufacturing process. In another example, stacks or rolls of dry wipers are packaged appropriately for consumers. The wetting composition is then added to the wipers shortly before use. The wetting composition may be automatically dispensed onto the wiper, or the wetting composition may be added manually. In this example there may be savings in shipping costs, and the shelf life of the product may be extended and/or the need for preservatives reduced.

Relative to the weight of the dry substrate, the wipe may desirably contain from about 10 percent to about 600 percent of the wetting composition, more desirably from about 50 percent to about 500 percent of the wetting composition, and even more desirably from about 100 percent to about 400 percent of the wetting composition. The wipe maintains its desired characteristics over the time periods involved in warehousing, transportation, retail display and storage by the consumer. Accordingly, shelf life may range from two months to two years. Various forms of impermeable envelopes and storage means for containing wet-packaged materials, such as wipes and towelettes and the like, are well known in the art. Any of these may be employed in packaging the wet wipes.

Desirably, the wet wipes are wetted with a wetting composition that is compatible with the ionic copolymer and any other components of the binder composition. The wetting composition also may enable the wet wipe to maintain its wet strength during converting, storage and usage (including dispensing), as well as, dispersibility in a toilet bowl. Additionally, the wetting composition may, by way of example, have some or all of the following characteristics: it does not cause skin irritation; it reduces tackiness of the wipe; it provides unique tactile properties such as skin glide and a “lotion-like feel”; and/or it acts as a vehicle to deliver “moist cleansing” and other skin health benefits.

A wetting composition for the fibrous substrates desirably contains an insolubilizing agent that maintains the strength of a water-dispersible binder composition until the insolubilizing agent is diluted with water, whereupon the strength of the water-dispersible binder composition begins to decay. The water-dispersible binder composition may be any of the binder compositions disclosed above. The insolubilizing agent in the wetting composition can be a salt, such as those disclosed for the various ionic copolymers, a blend of salts having both monovalent and multivalent ions, or any other compound, which provides in-use and storage strength to the water-dispersible binder composition, and can be diluted in water to permit dispersion of the substrate as the binder composition transitions to a weaker state. Desirably, the wetting composition contains more than about 0.3 weight percent of an insolubilizing agent based on the total weight of the wetting composition. Preferably, the wetting composition may contain from about 0.3 weight percent to about 10 weight percent of an insolubilizing agent. More preferably, the wetting composition may contain from about 0.5 weight percent to about 5 weight percent of an insolubilizing agent. More preferably, the wetting composition may contain from about 1 weight percent to about 4 weight percent of an insolubilizing agent.

The wetting composition may combine pharmaceuticals, such as nitroglycerine or scopolamine, and dimethyl sulfoxide (DMSO). The DMSO can accelerate the penetration of the pharmaceuticals through the skin. 100781 The wetting composition may further comprise a variety of additives compatible with the insolubilizing agent and the water-dispersible binder composition, such that the strength and dispersibility functions of the wipe are not jeopardized. Suitable additives in the wetting composition include, but are not limited to, the following additives: skin-care additives; odor control agents; detackifying agents to reduce the tackiness of the binder composition; particulates; antimicrobial agents; preservatives; wetting agents and cleaning agents, such as detergents, surfactants, some silicones; emollients; surface feel modifiers for improved tactile sensation (e.g., lubricity) on the skin; fragrance; fragrance solubilizers; opacifiers; fluorescent whitening agents; UV absorbers; pharmaceuticals; and pH control agents, such as malic acid or potassium hydroxide.

Skin-care additives include for example, enzyme inhibitors and sequestrants. The wetting composition may contain less than about 5 weight percent of skin-care additives based on the total weight of the wetting composition. More preferably, the wetting composition may contain from about 0.01 weight percent to about 2 weight percent of skin-care additives, more preferably from about 0.01 weight percent to about 0.05 weight percent of skin-care additives.

A variety of skin-care additives may be added to the wetting composition. The skin care additives may be organophilic clays such as described in U.S. Pat. No. 6,051,749, including reaction products of a long chain organic quaternary ammonium compound with one or more of the following clays: montmorillonite, bentonite, beidellite, hectorite, saponite, and stevensite. Other known enzyme inhibitors and sequestrants may be used as skin-care additives in the wetting composition, including those that inhibit trypsin and other digestive or fecal enzymes, and inhibitors for urease. Such inhibitors include transition metal ions and their soluble salts, such as silver, copper, zinc, ferric, and aluminum salts. The anion may also provide urease inhibition, such as borate, phytate, etc. Useful compounds include for example, silver chlorate, silver nitrate, mercury acetate, mercury chloride, mercury nitrate, copper metaborate, copper bromate, copper bromide, copper chloride, copper dichromate, copper nitrate, copper salicylate, copper sulfate, zinc acetate, zinc borate, zinc phytate, zinc bromate, zinc bromide, zinc chlorate, zinc chloride, zinc sulfate, cadmium acetate, cadmium borate, cadmium bromide, cadmium chlorate, cadmium chloride, cadmium formate, cadmium iodate, cadmium iodide, cadmium permanganate, cadmium nitrate, cadmium sulfate, and gold chloride.

Other salts that have been disclosed as having urease inhibition properties include ferric and aluminum salts, especially the nitrates, and bismuth salts. Other urease inhibitors include hydroxamic acid and its derivatives; thiourea; hydroxylamine; salts of phytic acid; extracts of plants of various species, including various tannins, e.g. carob tannin, and their derivatives such as chlorogenic acid derivatives; naturally occurring acids such as ascorbic acid, citric acid, and their salts; phenyl phosphoro diamidate/diamino phosphoric acid phenyl ester; metal aryl phosphoramidate complexes, including substituted phosphorodiamidate compounds; phosphoramidates without substitution on the nitrogen; boric acid and/or its salts, including especially, borax, and/or organic boron acid compounds; sodium, copper, manganese, and/or zinc dithiocarbamate; quinones; phenols; thiurams; substituted rhodanine acetic acids; alkylated benzoquinones; formarnidine disulphide; 1:3-diketones maleic anhydride; succinamide; phthalic anhydride; pehenic acid; N,N-dihalo-2-imidazolidinones; N-halo2-oxazolidinones; thio- and/or acyl- phosphorylamide and/or substituted derivatives thereof, thiopyridine-N-oxides, thiopyridines, and thiopyrimidines; oxidized sulfur derivatives of diaminophosphinyl compounds; cyclotriphosphazatriene derivatives; ortho-diaminophosphinyl derivatives of oximes; bromo-nitro compounds; S-aryl and/or alkyl diamidophosphorothiolates; diaminophosphinyl derivatives; mono- and/or polyphosphorodiamide; 5-substituted-benzoxathiol-2-ones; N(diaminophosphinyl)arylcarboxamides; and alkoxy-1,2-benzothaizin compounds.

Many other skin-care additives may be incorporated into the wetting composition, including for example, sun blocking agents and UV absorbers; acne treatments; pharmaceuticals; baking soda, including encapsulated forms thereof; vitamins and their derivatives such as Vitamins A or E; botanicals such as witch hazel extract and aloe vera; allantoin; emollients; disinfectants; hydroxy acids for wrinkle control or anti-aging effects; sunscreens; tanning promoters; skin lighteners; deodorants and anti-perspirants; ceramides for skin benefits and other uses; astringents; moisturizers; nail polish removers; insect repellants; antioxidants; antiseptics; and anti-inflammatory agents; provided that the additives are compatible with an ion-sensitive binder composition associated therewith.

Suitable odor control additives for use in the wetting composition and wet wipes include, for example, zinc salts; talc powder; encapsulated perfumes, including microcapsules, macrocapsules, and perfume encapsulated in liposomes, vessicles, or microemulsions; chelants, such as ethylenediamine tetra-acetic acid; zeolites; activated silica, activated carbon granules or fibers; activated silica particulates; polycarboxylic acids, such as citric acid; cyclodextrins and cyclodextrin derivatives; chitosan or chitin and derivatives thereof; oxidizing agents; antimicrobial agents, including silver-loaded zeolites (e.g., those of BF Technologies, located in Beverly, Mass., sold under the trademark HEALTHSHIELD™); triclosan; kieselguhr; and mixtures thereof. In addition to controlling odor from the body or body wastes, odor control strategies can also be employed to mask or control any odor of the treated substrate. Preferably, the wetting composition contains less than about 5 weight percent of odor control additives based on the total weight of the wetting composition. More preferably, the wetting composition contains from about 0.01 weight percent to about 2 weight percent of odor control additives, more preferably from about 0.03 weight percent to about 1 weight percent of odor control additives. The wetting composition and/or wet wipes may comprise derivatized cyclodextrins, such as hydroxypropyl beta-cyclodextrin in solution, which remain on the skin after wiping and provide an odor-absorbing layer. Alternatively, the odor source may be removed or neutralized by application of an odor-control additive, exemplified by the action of a chelant that binds metal groups necessary for the function of many proteases and other enzymes that commonly produce an odor.

Detackifying agents may be used in the wetting composition to reduce the tackiness, if any, of the binder composition. Suitable detackifiers include any substance known in the art to reduce tack between two adjacent fibrous sheets treated with an adhesive-like polymer or any substance capable of reducing the tacky feel of an adhesive-like polymer on the skin. Specific detackifiers include for example, powders, such as talc powder, calcium carbonate, and mica; starches, such as corn starch; lycopodium powder; mineral fillers, such as titanium dioxide; silica powder; alumina; metal oxides in general; baking powder; and kieselguhr. Polymers and other additives having low surface energy may also be used, including for example a wide variety of fluorinated polymers; silicone additives; polyolefins and thermoplastics; waxes; and debonding agents known in the paper industry including compounds having alkyl side chains such as those having 16 or more carbons. Release agents for molds and candle making may also be used, as well as dry lubricants and fluorinated release agents. The wetting composition preferably contains less than about 25 weight percent of detackifying agents based on the total weight of the wetting composition. More preferably, the wetting composition contains from about 0.01 weight percent to about 10 weight percent of detackifying agents, more preferably about 5% or less, more preferably from about 0.05 weight percent to about 2 weight percent of detackifying agents.

The wetting composition may be further modified by the addition of solid particulates or microparticulates. Suitable particulates include for example, mica, silica, alumina, calcium carbonate, kaolin, talc, and zeolites. The particulates may be treated with stearic acid or other additives to enhance the attraction or bridging of the particulates to the binder composition, if desired. Also, two-component microparticulate systems, commonly used as retention aids in the papermaking industry, may also be used. Such two-component microparticulate systems typically comprise a colloidal particle phase, such as silica particles, and a water-soluble cationic polymer for bridging the particles to the fibers of the web to be formed. Preferably, the wetting composition contains less than about 25 weight percent of particulate based on the total weight of the wetting composition. More preferably, the wetting composition may contain from about 0.05 weight percent to about 10 weight percent of microparticulate, more preferably from about 0.1 weight percent to about 5 weight percent of microparticulate.

Microcapsules and other delivery vehicles may also be used in the wetting composition to provide skin-care agents; medications; comfort promoting agents, such as eucalyptus; perfumes; skin care agents; odor control additives; vitamins; powders; and other additives to the skin of the user. Preferably, the wetting composition may contain up to about 25 weight percent of microcapsules or other delivery vehicles based on the total weight of the wetting composition. More preferably, the wetting composition may contain from about 0.05 weight percent to about 10 weight percent of microcapsules or other delivery vehicles, more preferably from about 0.2 weight percent to about 5.0 weight percent of microcapsules or other delivery vehicles. Microcapsules and other delivery vehicles are well known in the art. For example, POLY-PORE® E200 (Chemdal Corp., Arlington Heights, Ill.), POLY-POREO L200, cyclodextrins and their derivatives, liposomes, polymeric sponges, and spray-dried starch. Additives present in microcapsules are isolated from the environment and the other agents in the wetting composition until the wipe is applied to the skin, whereupon the microcapsules break and deliver their load to the skin or other surfaces.

The wetting composition may also contain preservatives and/or anti-microbial agents. Several preservatives and/or anti-microbial agents, such as Mackstat H 66 (available from McIntyre Group, Chicago, Ill.), have been found to give excellent results in preventing bacteria and mold growth. Other suitable preservatives and anti-microbial agents include for example DMDM hydantoin (e.g., Glydant Plus™, Lonza, Inc., Fair Lawn, N.J.); iodopropynyl butylcarbamate; Kathon (Rohm and Hass, Philadelphia, Pa.); methylparaben; propylparaben; 2-bromo-2-nitropropane-1,3-diol; and benzoic acid. Preferably, the wetting composition contains less than about 2 weight percent on an active basis of preservatives and/or anti-microbial agents based on the total weight of the wetting composition. More preferably, the wetting composition contains from about 0.01 weight percent to about 1 weight percent of preservatives and/or anti-microbial agents, more preferably from about 0.01 weight percent to about 0.5 weight percent of preservatives and/or anti-microbial agents.

A variety of wetting agents and/or cleaning agents may be used in the wetting composition. Suitable wetting agents and/or cleaning agents include for example, detergents and nonionic, amphoteric, and anionic surfactants, such as amino acid-based surfactants. One function of the surfactant is to improve wetting of the dry substrate with the wetting composition. Another function of the surfactant can be to disperse bathroom soils when the wet wipe contacts a soiled area and to enhance their absorption into the substrate. The surfactant can further assist in make-up removal, general personal cleansing, hard surface cleansing, odor control, and the like. One commercial example of an amino-acid based surfactant is acylglutamate, marketed under the Amisoft name by Ajinomoto Corp., Tokyo, Japan. Preferably, the wetting composition contains less than about 3 weight percent of wetting agents and/or cleaning agents based on the total weight of the wetting composition. More preferably, the wetting composition contains from about 0.01 weight percent to about 2 weight percent of wetting agents and/or cleaning agents, more preferably from about 0.1 weight percent to about 0.5 weight percent of wetting agents and/or cleaning agents.

A wide variety of surfactants may be used. Non-ionic surfactants include for example, the condensation products of ethylene oxide with a hydrophobic (oleophilic) polyoxyalkylene base formed by the condensation of propylene oxide with propylene glycol, for example pluronic surfactants (BASF Wyandotte Corp.), such as Pluronic L-62. Other useful nonionic surfactants include for example, the condensation products of C₈-C₂₂ alkyl alcohols with 2-50 moles of ethylene oxide per mole of alcohol. Examples of compounds of this type include the condensation products of C₁₁-C₁₅ secondary alkyl alcohols with 3-50 moles of ethylene oxide per mole of alcohol, which are commercially-available as the Poly-Tergent SLF series from Olin Chemicals or the TERGITOL® series from Union Carbide, i.e. TERGITOL® 25-L-7. Other nonionic surfactants include the ethylene oxide esters of C₆-C₁₂ alkyl phenols such as (nonylphenoxy)-polyoxyethylene ether, for example the IGEPAL® CO series (GAF Corp.). Further non-ionic surface active agents include for example, alkyl polyglycosides (APG), derived as a condensation product of dextrose (D-glucose) and a straight or branched chain alcohol, such as those available from Horizon Chemical under the trade names of APG-300, APG-350, APG-500, and APG-500. Silicones are another class of wetting agents available in pure form, or as microemulsions, macroemulsions, and the like. One exemplary non-ionic surfactant group is the silicone-glycol copolymers, available from the Dow Coming Corp as Dow Coming 190 and 193 surfactants (CTFA name: dimethicone copolyol).

Anionic surfactants may also be used in the wetting compositions, including anionic detergent salts having alkyl substituents of 8 to 22 carbon atoms such as the water-soluble higher fatty acid alkali metal soaps, e.g., sodium myristate and sodium palmitate; and water-soluble sulfated and sulfonated anionic alkali metal and alkaline earth metal detergent salts containing a hydrophobic higher alkyl moiety (typically containing from about 8 to 22 carbon atoms) such as salts of higher alkyl mono or polynuclear aryl sulfonates having from about 1 to 16 carbon atoms in the alkyl group, with examples available as the Bio-Soft series, i.e. Bio-Soft D-40 (Stepan Chemical Co.). Other useful classes of anionic surfactants include for example, the alkali metal salts of alkyl naphthalene sulfonic acids (methyl naphthalene sodium sulfonate, Petro AA, Petrochemical Corporation); sulfated higher fatty acid monoglycerides such as the sodium salt of the sulfated monoglyceride of cocoa oil fatty acids and the potassium salt of the sulfated monoglyceride of tallow fatty acids; alkali metal salts of sulfated fatty alcohols containing from about 10 to 18 carbon atoms (e.g., sodium lauryl sulfate and sodium stearyl sulfate); sodium C₁₄-C₁₆-alphaolefin sulfonates such as the Bio-Terge series (Stepan Chemical Co.); alkali metal salts of sulfated ethyleneoxy fatty alcohols (the sodium or ammonium sulfates of the condensation products of about 3 moles of ethylene oxide with a C₁₂-C₁₅ n-alkanol, i.e., the Neodol ethoxysulfates, Shell Chemical Co.); alkali metal salts of higher fatty esters of low molecular weight alkylol sulfonic acids, e.g. fatty acid esters of the sodium salt of isothionic acid, the fatty ethanolamide sulfates; the fatty acid amides of amino alkyl sulfonic acids, e.g. lauric acid amide of taurine; as well as numerous other anionic organic surface active agents such as sodium xylene sulfonate, sodium naphthalene sulfonate, sodium toulene sulfonate and mixtures thereof. Other useful anionic surfactants include sodium cocoyl glutamate, TEA cocoyl glutamate, and sodium cocoyl sarcosinate. A further useful class of anionic surfactants includes the 8-(4-n-alkyl-2-cyclohexenyl)-octanoic acids, wherein the cyclohexenyl ring is substituted with an additional carboxylic acid group. These compounds or their potassium salts, are commercially-available from Westvaco Corporation as Diacid 1550 or H-240. In general, these anionic surface active agents can be employed in the form of their alkali metal salts, ammonium or alkaline earth metal salts.

The wetting composition may further comprise an aqueous microemulsion of silicone particles, for example as described in U.S. Pat. No. 6,037,407. Preferably, the wetting composition contains less than about 5 weight percent of a microemulsion of silicone particles based on the total weight of the wetting composition. More preferably, the wetting composition contains from about 0.02 weight percent to about 3 weight percent of a microemulsion of silicone particles, more preferably from about 0.02 weight percent to about 0.5 weight percent of a microemulsion of silicone particles. For example, the wetting composition may comprise a silicone copolyol sulfosuccinate, such as disodium dimethicone copolyol sulfosuccinate and diammonium dimethicone copolyolsulfosuccinate. Preferably, the wetting composition comprises less than about 2 percent by weight of the silicone-based sulfosuccinate, and more preferably from about 0.05 percent to about 0.30 percent by weight of the silicone-based sulfosuccinate. In another example of a product comprising a silicone emulsions, Dow Corning 9506 powder may also be present in the wetting composition.

The wetting composition may also contain one or more emollients. Suitable emollients include for example, PEG 75 lanolin; methyl gluceth 20 benzoate; C₁₂-C₁₅ alkyl benzoate; ethoxylated cetyl stearyl alcohol; products marketed as Lambent wax WS-L, Lambent WD-F, Cetiol HE (Henkel Corp.), Glucam P20 (Amerchol), Polyox WSR N-10 (Union Carbide), Polyox WSR N-3000 (Union Carbide), Luviquat (BASF), Finsolv SLB 101 (Finetex Corp.), Estol 1517 (Unichema), and Finsolv SLB 201 (Finetex Corp.); mink oil; allantoin; and stearyl alcohol. The emollient composition in such products and other useful products can comprise a plastic or fluid emollient such as one or more liquid hydrocarbons (e.g., petrolatum), mineral oil and the like; vegetable and animal fats (e.g., lanolin, phospholipids and their derivatives); and/or silicone materials such as one or more alkyl substituted polysiloxane polymers, including the polysiloxane emollients disclosed in U.S. Pat. No. 5,891,126. Optionally, a hydrophilic surfactant may be combined with a plastic emollient to improve wettability of the coated surface. Liquid hydrocarbon emollients and/or alkyl substituted polysiloxane polymers may be blended or combined with one or more fatty acid ester emollients derived from fatty acids or fatty alcohols.

The emollient material may be in the form of an emollient blend. Preferably, the emollient blend comprises a combination of one or more liquid hydrocarbons (e.g., petrolatum), mineral oil and the like; vegetable and animal fats (e.g., lanolin, phospholipids and their derivatives); with a silicone material such as one or more alkyl substituted polysiloxane polymers. More preferably, the emollient blend comprises a combination of liquid hydrocarbons (e.g., petrolatum) with dimethicone, or with dimethicone and other alkyl substituted polysiloxane polymers. Blends of liquid hydrocarbon emollients and/or alkyl substituted polysiloxane polymers may be blended with one or more fatty acid ester emollients derived from fatty acids or fatty alcohols, or with PEG-7 glyceryl cocoate, available as Standamul HE (Henkel Corp., Hoboken, N.J).

Water-soluble, self-emulsifying emollient oils include the polyoxyalkoxylated lanolins and the polyoxyalkoxylated fatty alcohols, as disclosed in U.S. Pat. No. 4,690,821. The polyoxyalkoxy chains preferably comprise mixed propylenoxy and ethyleneoxy units. The lanolin derivatives will typically comprise about 20-70 such lower-alkoxy units, while the C₁₂-C₂₀ fatty alcohols are derivatized with about 8-15 lower- alkyl units. One such useful lanolin derivative is Lanexol AWS (PPG-12-PEG-50, Croda, Inc., New York, N.Y.). A useful poly(15-20)C₂-C₃-alkoxylate is PPG-5-Ceteth-20, known as Procetyl AWS (Croda, Inc.).

Preferably, the wetting composition contains less than about 25 weight percent of emollients based on the total weight of the wetting composition. More preferably, the wetting composition comprises less than about 5 weight percent emollient, more preferably less than about 2% emollient, more preferably from about 0.01 weight percent to about 8 weight percent of emollients, more preferably from about 0.2 weight percent to about 2 weight percent of emollients. The wetting composition and/or wet wipes can comprise an oil-in-water emulsion comprising an oil phase containing at least one emollient oil and at least one emollient wax stabilizer dispersed in an aqueous phase comprising at least one polyhydric alcohol emollient and at least one organic water-soluble detergent, as disclosed in U.S. Pat. No. 4,559,157.

Surface feel modifiers may be used to improve the tactile sensation (e.g., lubricity) of the skin during use of the product. Suitable surface feel modifiers include, for example, commercial debonders; and softeners, such as the softeners used in the art of tissue making including quaternary ammonium compounds with fatty acid side groups, silicones, waxes, and the like. Exemplary quaternary ammonium compounds with utility as softeners are disclosed in U.S. Pat. Nos. 3,554,862; 4,144,122; 5,573,637; and 4,476,323. Preferably, the wetting composition contains less than about 2 weight percent of surface feel modifiers based on the total weight of the wetting composition. More preferably, the wetting composition contains from about 0.01 weight percent to about 1 weight percent of surface feel modifiers, more preferably from about 0.01 weight percent to about 0.05 weight percent of surface feel modifiers.

A variety of fragrances may be used in the wetting composition of the present invention. Desirably, the wetting composition contains less than about 2 weight percent of fragrances based on the total weight of the wetting composition. More desirably, the wetting composition contains from about 0.01 weight percent to about 1 weight percent of fragrances. Even more desirably, the wetting composition contains from about 0.01 weight percent to about 0.05 weight percent of fragrances.

Further, a variety of fragrance solubilizers may be used in the wetting composition of the present invention. Suitable fragrance solubilizers include, but are not limited to, polysorbate 20, propylene glycol, ethanol, isopropanol, diethylene glycol monoethyl ether, dipropylene glycol, diethyl phthalate, triethyl citrate, Ameroxol OE-2 (Amerchol Corp.), Brij 78 and Brij 98 (ICI Surfactants), Arlasolve 200 (ICI Surfactants), Calfax 16L-35 (Pilot Chemical Co.), Capmul POE-S (Abitec Corp.), Finsolv SUBSTANTIAL (Finetex), and the like. Desirably, the wetting composition contains less than about 2 weight percent of fragrance solubilizers based on the total weight of the wetting composition. More desirably, the wetting composition contains from about 0.01 weight percent to about 1 weight percent of fragrance solubilizers. Even more desirably, the wetting composition contains from about 0.01 weight percent to about 0.05 weight percent of fragrance solubilizers.

Examples of opacifiers include titanium dioxide or other minerals or pigments, and synthetic opacifiers such as REACTOPAQUE® particles (available from Sequa Chemicals, Inc., Chester, S.C.). Preferably, the wetting composition contains less than about 2 weight percent of opacifiers based on the total weight of the wetting composition, more preferably from about 0.01 weight percent to about 1 weight percent of opacifiers, more preferably from about 0.01 weight percent to about 0.05 weight percent of opacifiers.

Examples of pH control agents for use in the wetting composition include malic acid, citric acid, hydrochloric acid, acetic acid, sodium hydroxide, potassium hydroxide, and the like. Preferably, the pH range of the wetting composition is from about 3.5 to about 6.5, more preferably from about 4 to about 6. Preferably, the wetting composition contains less than about 2 weight percent of a pH adjuster based on the total weight of the wetting composition, more preferably from about 0.01 weight percent to about 1 weight percent of a pH adjuster, more preferably from about 0.01 weight percent to about 0.05 weight percent of a pH adjuster.

A variety of wetting compositions, formed from water, a salt, at least about 10 wt % organic solvent, and optionally one or more of the other above-described additives, may be used with the wet wipes. In one example, the wetting composition contains the following components, given in weight percent of the wetting composition, as shown in Table 2 below:

TABLE 2
Wetting Composition Components

| Wetting Composition Component: | Weight Percent: |
|---|---|
| Deionized Water | About 50 to about 90 |
| Salt | About 0.3 to about 20 |
| Organic solvent | About 10 to about 50 |
| Preservative | Up to about 2 |
| Surfactant | Up to about 2 |
| Silicone Emulsion | Up to about 1 |
| Emollient | Up to about 1 |
| Fragrance | Up to about 0.3 |
| Fragrance solubilizer | Up to about 0.5 |
| pH adjuster | Up to about 0.2 |

In another example, the wetting composition comprises the following components, given in weight percent of the wetting composition, as shown in Table 3 below:

TABLE 3
Wetting Composition Components

| Class of | | | |
|---|---|---|---|
| Wetting | Specific Wetting | | |
| Composition | Composition | Component | |
| Component: | Component: | Name: | Weight Percent: |
| Vehicle | Deionized Water | | about 50 to about |
| | | | 90 |
| Salt | Sodium Chloride | | about 0.3 to about |
| | (Millport Ent., | | 20 |
| | Milwaukee, WI) | | |
| Organic | Ethanol | | About 10 to about |
| solvent | | | 50 |
| Preservative | Glycerin, IPBC and | Mackstat H-66 | Up to about 2 |
| | DMDM Hydantoin | (McIntyre | |
| | | Group, | |
| | | Chicago, IL) | |
| Surfactant | Acyl Glutamate | CS22 | Up to about 2 |
| | | (Ajinomoto, | |
| | | Tokyo, Japan) | |
| Silicone | Dimethiconol and | DC1785 | Up to about 1 |
| Emulsion | TEA | (Dow Corning, | |
| (Detackifier/ | Dodecylbenezene | Midland, MI) | |
| Skin Feel | Sulfonate | | |
| agent) | | | |
| Emollient | PEG-75 Lanolin | Solulan L- 575 | Up to about 1 |
| | | (Amerchol, | |
| | | Middlesex, NJ) | |
| Fragrance | Fragrance | Dragoco | Up to about 0.3 |
| | | 0/708768 | |
| | | (Dragoco, | |
| | | Roseville, MN) | |
| Fragrance | Polysorbate 20 | Glennsurf L20 | Up to about 0.5 |
| solubilizer | | (Glenn Corp., | |
| | | St. Paul, MN) | |
| pH adjuster | Malic Acid to pH 5 | | Up to about 0.2 |
| | (Haarman & Reimer, | | |
| | Tetrboro, NJ) | | |

In another example, the wetting composition comprises the following components, given in weight percent of the wetting composition, as shown in Table 4 below:

TABLE 4
An Exemplary Wetting Composition

| Class of | | | |
|---|---|---|---|
| Wetting | Specific Wetting | | |
| composition | composition | Component | |
| Component: | Component: | Name: | Weight Percent: |
| Vehicle | Deionized Water | | about 73 |
| Insolubilizing | Sodium Chloride | | about 4 |
| compound | | | |
| Organic | Ethanol | | About 20 |
| solvent | | | |
| Preservative | Glycerin, IPBC and | Mackstat | about 1 |
| | DMDM Hydantoin | H-66 | |
| Surfactant | Acyl Glutamate | CS22/ECS 22P | about 1 |
| Silicone | Dimethiconol and | DC 1784/ | about 0.5 |
| Emulsion | TEA | DC1785 | |
| | Dodecylbenezene | | |
| | Sulfonate | | |
| Emollient | PEG-75 Lanolin | Solulan L- 575 | about 0.25 |
| Fragrance | Fragrance | Dragoco | about 0.05 |
| | | Fragrance | |
| | | 0/708768 | |
| Fragrance | Polysorbate 20 | Glennsurf L20 | about 0.25 |
| solubilizer | | | |
| pH adjuster | Malic Acid to pH 5 | | about 0.07 |

It should be noted that the above-described wetting compositions may be used with any one of the above-described binder compositions containing an ionic copolymer. Further, the above-described wetting compositions may be used with any other binder composition, including conventional binder compositions, or with any known fibrous or absorbent substrate, whether dispersible or not.

In one example, wet wipes may be produced using the wetting composition described in Table 3 and an air-laid fibrous substrate comprising about 75 weight percent of bleached kraft fibers and 25 weight percent of any of the above-described binder composition, wherein the weight percentages are based on the total weight of the dry nonwoven fibrous substrate. The amount of wetting composition added to the nonwoven fibrous substrate, relative to the dry weight of the nonwoven fibrous substrate in these examples, is desirably about 180 percent to about 240 weight percent. In a further example, water-dispersible wet wipes may be produced using the above-described wetting composition in Table 2 and an air-laid fibrous substrate comprising 80 weight percent of softwood fibers and 20 weight percent of the binder composition. The amount of wetting composition added to the nonwoven fibrous substrate, relative to the dry weight of the nonwoven fibrous substrate in these examples, is desirably about 180 percent to about 240 weight percent. In a further example, water-dispersable wet wipes may be produced using the wetting composition described in Table 2 and an air-laid fibrous substrate comprising 90 weight percent of softwood fibers and 10 weight percent of the binder composition. The amount of wetting composition added to the nonwoven fibrous substrate, relative to the dry weight of the nonwoven fibrous substrate in these examples, is desirably about 180 percent to about 240 weight percent.

Desirably, the wet wipes possess an in-use wet tensile strength of at least about 100 g/in when soaked with 10% to 400% by weight wet wipes solution containing more than 0.5% by weight monovalent and/or divalent salts, such as NaCi, ZnCl₂ and/or CaCl₂ or mixtures thereof, and a tensile strength of less than about 30 g/in after being soaked in soft water or hard water containing up to 200 ppm concentration of Ca²⁺ and/or Mg²⁺ for 24 hours or less, preferably after about one hour. For handsheet substrates, cross deckle wet tensile strengths (CDWT) have been reported. Machine direction wet tensile strengths (MDWT) have been reported for substrates made on a continuous former.

More desirably, the wet wipes possess an in-use wet tensile strength of at least about 300 g/in when soaked with 10% to 400% by weight wet wipes solution containing more than 0.5% by weight monovalent and/or divalent salts, such as NaCl, ZnCl₂ and/or CaCl₂ or mixtures thereof, and a tensile strength of less than about 75 g/in after being soaked in soft water or hard water containing up to 200 ppm concentration of Ca²⁺ and/or Mg²⁺ for 24 hours or less, preferably after about one hour.

Most desirably, the wet wipes possess an in-use wet tensile strength of >300 g/in when soaked with 10% to 400% by weight wet wipes solution containing more than 0.5% by weight monovalent and/or divalent salts, such as NaCl, ZnCl₂ and/or CaCl₂ or mixtures thereof, and a tensile strength of less than about 30 g/in after being soaked in soft water or hard water containing up to 200 ppm concentration of Ca²⁺ and/or Mg²⁺ for 24 hours or less, preferably after about one hour.

Products with higher basis weights than flushable wet wipes may have relatively higher wet tensile strength. For example, products, such as wet towels or hard-surface cleaning wipes, may have basis weights above 70 gsm, such as from 80 gsm to 150 gsm. Such products can have CDWT values of 500 g/in or greater, and after soaking values of about 150 g/in or less, more specifically about 100 g/in or less, and most specifically about 50 g/in or less.

The wet wipes can be made in several ways. In one example, the binder composition is applied to a fibrous substrate as part of an aqueous solution or suspension, wherein subsequent drying is needed to remove the water and promote binding of the fibers. In particular, during drying, the binder composition migrates to the crossover points of the fibers and becomes activated as a binder in those regions, thus providing acceptable strength to the substrate. For example, the following steps can be applied:

1. Providing an absorbent substrate that is not highly bonded (e.g., an unbonded airlaid, a tissue web, a carded web, fluff pulp, etc.).

2. Applying a binder composition to the substrate, typically in the form of a liquid, suspension, or foam.

3. Drying the substrate to promote bonding of the substrate. The substrate may be dried such that the peak substrate temperature does not exceed about 100° to 220° C.

5. Applying a wetting composition to the substrate.

6. Placing the wetted substrate in roll form or in a stack and packaging the product.

Application of the binder composition to the substrate can be by means of spray; by foam application; by immersion in a bath; by curtain coating; by coating and metering with a wire-wound rod; by passage of the substrate through a flooded nip; by contact with a pre-metered wetted roll coated with the binder solution; by pressing the substrate against a deformable carrier containing the binder composition such as a sponge or felt to effect transfer into the substrate; by printing such as gravure, inkjet, or flexographic printing; and any other means known in the art.

In the use of foams to apply a binder composition, the mixture is frothed, typically with a foaming agent, and spread uniformly on the substrate, after which vacuum is applied to pull the froth through the substrate. Any known foam application method can be used, including that of U.S. Pat. No. 4,018,647, “Process for the Impregnation of a Wet Fiber Web with a Heat Sensitized Foamed Latex Binder,” issued Apr. 19, 1977 to Wietsma, the entirety of which is herein incorporated by reference. Wietsma discloses a method wherein a foamed latex is heat-sensitized by the addition of a heat-sensitizer such as functional siloxane compounds including siloxane oxyalkylene block copolymers and organopolysiloxanes. Specific examples of applicable heat-sensitizers and their use thereof for the heat sensitization of latices are described in the U.S. Pat. Nos. 3,255,140; 3,255,141; 3,483,240 and 3,484,394, all of which are incorporated herein by reference. The use of a heat-sensitizer is said to result in a product having a very soft and textile-like hand compared to prior methods of applying foamed latex binders.

The amount of heat-sensitizer to be added is dependent on factors including the type of latex used, the desired coagulation temperature, the machine speed and the temperatures in the drying section of the machine, and will generally be in the range of about 0.05 to about 3% by weight, calculated as dry matter on the dry weight of the latex; but also larger or smaller amounts may be used. The heat sensitizer can be added in such an amount that the latex will coagulate far below the boiling point of water, for instance at a temperature in the range of 35° C. to 95° C., or from about 35° C. to 65° C.

Without wishing to be bound by theory, it is believed that a drying step after application of the binder solution and before application of the wetting composition enhances bonding of a fibrous substrate by driving the binder composition to fiber crossover points as moisture is driven off, thus promoting efficient use of the binder. However, in an alternative method, the drying step listed above is skipped, and the binder composition is applied to the substrate followed by application of the wetting composition without significant intermediate drying. In one version of this method, the binder composition selectively adheres to the fibers, permitting excess water to be removed in an optional pressing step without a significant loss of the binder composition from the substrate. In another version, no significant water removal occurs prior to application of the wetting composition. In yet another alternative method, the binder composition and the wetting composition are applied simultaneously, optionally with subsequent addition of salt or other insolubilizing compounds to further render the binder composition insoluble.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Binder Composition Containing Cationic Copolymer

A cationic acrylate polymer was synthesized in a 75/25 acetone/water mixture at approximately 30% total monomer solids. Vazo-52 (DuPont) was utilized as a free-radical initiator. Acetone (VWR, Westchester, Pa.) 399 g and deionized (DI) water, 125 g, were charged into a 3 L four-neck round bottom flask. The flask was cooled in an ice bath and bubbled with nitrogen for 20 minutes to eliminate oxygen. The reaction flask was heated to reflux (approximately 60° C.) prior to adding the monomer feeds and kept under nitrogen during reaction. ADAMQUAT MC-80 (Atofina Chemicals, Philadelphia, Pa.), 39.6 g, was diluted with 42.0 g of DI water and bubbled with nitrogen as it was fed into the reaction flask. Methyl acrylate (Atofina Chemicals, Philadelphia, Pa.), 267.7 g, and Vazo-52, 0.6 g, were dissolved in 126.1 g of acetone. This solution was cooled in an ice bath and bubbled with nitrogen as it was fed into the reaction flask. Monomer solutions were fed into the reaction flask over a period of 4 hours using mechanical dosing pumps and held at reflux for an additional 2 hours. The acetone was removed by distillation over a period of approximately 5 hours, adding DI water as the acetone was removed. An aqueous solution with approximately 0.2% residual acetone at about 23% solids was obtained.

### Example 2

### Binder Composition Containing Anionic Copolymer

An anionic copolymer was prepared by the solution polymerization of a mixture of monomers. The mixture contained 4 mole percent 2-acrylamido-2-methyl-1-propanesulfonic acid (AMPS), 22.5 mole percent butyl acrylate (BA) and 10.5 mole percent 2-ethylhexyl acrylate (EHA). The monomers were dissolved in a 90:10 mixture of acetone and water, and free radical polymerization was conducted at a temperature of 55-58° C. in the absence of chain transfer agent. The initiator was Vazo-52, which was present at a concentration of 0.3 mole percent relative to the total monomers. Total polymer solids during the reaction ranged between 20 wt % and 25 wt %. The temperature of the reaction mixture was maintained for 6 hours, after which an equimolar amount of sodium hydroxide relative to the AMPS was charged to the mixture. Mixing was continued for an additional 1 hour, and the reaction mixture was then concentrated by rotary evaporation.

An anionic binder composition was prepared by combining this anionic copolymer with poly(ethylene-vinyl acetate) (EVA) as a co-binder polymer. The EVA was Dur-O-Set® RB (National Starch and Chemical Co., Bridgewater, N.J.). The binder contained 75 wt % anionic copolymer and 25 wt % EVA.

### Example 3

### Formation of Fibrous Substrates

A weak, thermally-bonded air-laid (TBAL) nonwoven test substrate was fabricated from Weyerhauser NF405 wood pulp and KoSA T-255 binder fibers. The binder fiber had a polyester core and a polyethylene sheath that melts at approximately 130° C. The air-laid web was formed using approximately 4% binder fiber and thermally bonded above the melting temperature of the sheath. The TBAL basesheet had an average basis weight of 51 gsm and an average caliper of 1.0 mm.

A uniform and consistent amount of the binder composition of Example 1 or Example 2 was applied to the substrate via a pressurized spray unit. This handsheet spray unit was designed to closely resemble the operation of a commercial airlaid machine using liquid or emulsion binders, but on a much smaller scale. The equipment was enclosed in a small-framed housing, which could be placed under a laboratory hood. The unit had a stationary sample holder (10″×13″) in the center of the unit and a moveable spray header directly over the sample holder. A vacuum box was installed under the sample holder section to help draw the binder composition into the web during the application process. The hand-sheet was placed on the vacuum box and the spray head was moved across the substrate as the binder composition was sprayed in a flat V-shaped pattern. The binder composition was contained in a pressurized storage vessel located outside of the spray cabinet and was delivered to the spray nozzles via high pressure flexible tubing. The spray header with its spray nozzle (Spraying Systems Company) assembly was moved over the sample by means of a belt driven slide assembly, providing the desired application uniformity and speed. The spray header could be operated at speeds close to 180 fpm and the spray atomization pressure could be set as high as 200 psig. For a given binder composition, approximately one half of the desired binder composition add-on was sprayed on one side, the sheet was manually turned over, and the remaining binder composition add-on was applied to the second side. The fibrous substrate was removed from the vacuum box and dried in a Werner Mathis, Model LTV Through-Air Dryer (TAD), at a temperature of 193° C. for 23 seconds. Final basis weight of the samples with binder composition was approximately 63-64 gsm.

### Example 4

### Wet Wipes In-Use Strength: Effect Of Salt Concentration

The fibrous substrates of Example 3 were converted into wet wipes by applying to a sheet of the desired dimensions one of a number of different wetting compositions via a hand-held aerosol or pump-action sprayer. The binder composition for all of the fibrous substrates was the cationic binder of Example 1. The wetting compositions were DI water, aqueous saline solutions containing variable amounts of NaCl (0-4.0 wt %), and aqueous solutions containing variable amounts of organic solvent (0-10 wt %) and of NaCl (0-4.0 wt %). The specific type and amount of organic solvent and amount of NaCl is noted in Table 5 below. Add-on of the wetting composition was approximately 600% relative to the weight of the dry substrate.

The in-use strength was assessed by measuring the wet tensile strength of the substrate after application of the wetting composition. A SinTech 1/D tensile tester with Testworks 3.03 version software was used for all sample testing. A 100 Newton load cell with pneumatic grips was utilized. A gauge length of 2 in. and a crosshead speed of 12 in./min. were employed. The peak load values (g/in.) of sample replicates were recorded, averaged and reported as machine direction wet tensile strength (MDWT). For samples that were too weak to be handled and measured (typically less than 20 g/in.) a “0” was recorded for the peak load.

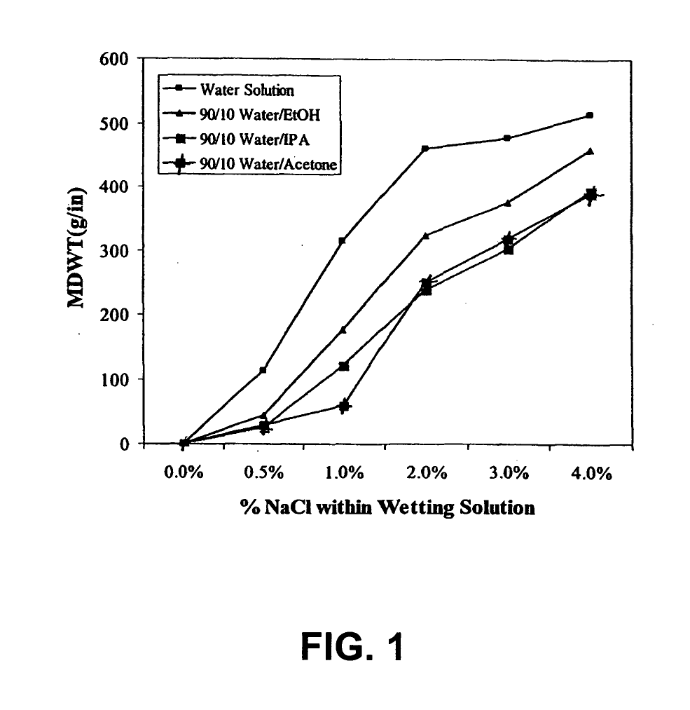

Table 5 lists the measured MDWT values for wet wipes having a wetting composition containing either no organic solvent, or containing 10 wt % of ethanol, iso-propanol or acetone. The data from this table are illustrated graphically in FIG. 1. MDWT values of 300-325 g-force/in were obtained with 1.0 wt % salt in the absence of organic solvent, with 2.0 wt % salt in the presence of 10 wt % ethanol, and with 3.0 wt % salt in the presence of 10 wt % iso-propanol or acetone.

TABLE 5
In-Use Strength of Wet Wipes With Variable Salt Concentration

MDWT (g-force/in width)
Organic Solvent In Wetting Composition

| wt % | | 10 wt % | 10 wt % | 10 wt % |
|---|---|---|---|---|
| NaCl | None | ethanol | iso-propanol | acetone |
| 0.0 | 0 | 0 | 0 | 0 |
| 0.5 | 113 | 43 | 26 | 29 |
| 1.0 | 314 | 177 | 121 | 58 |
| 2.0 | 460 | 322 | 238 | 250 |
| 3.0 | 477 | 374 | 303 | 317 |
| 4.0 | 514 | 459 | 393 | 388 |

### Example 5

### Wet Wipes In-Use Strength: Comparison Of Binders

The fibrous substrates of Example 3 were converted into wet wipes as described in Example 4. The binder composition for the fibrous substrates was either the cationic binder of Example 1, or the anionic binder of Example 2. The wetting compositions were aqueous saline solutions containing 4 wt % NaCl and either no organic solvent or 10 wt % organic solvent (ethanol, iso-propanol, or acetone). The in-use strength of each type of wet wipe was measured as described in Example 4.

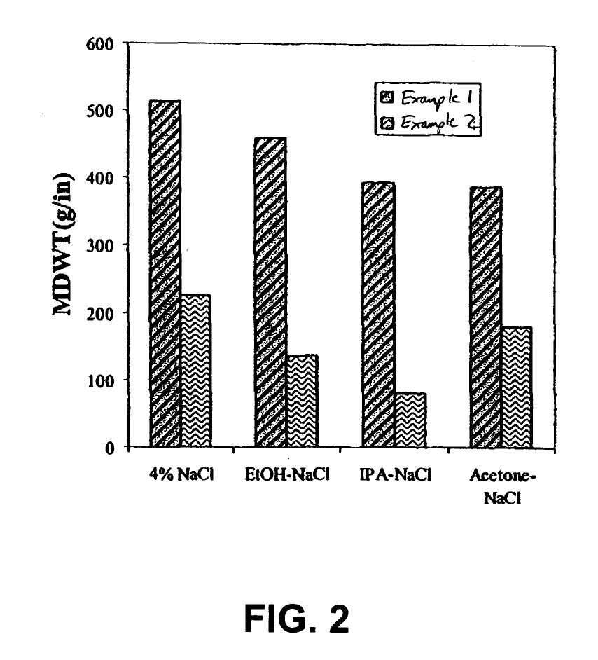

Table 6 lists the measured MDWT values for each type of substrate and wetting composition. This table also lists the percentage decrease in MDWT for the wet wipes containing organic solvent compared to wet wipes containing only 4 wt % aqueous NaCl. The data from this table are illustrated graphically in FIG. 2. The cationic binder of Example 1, characterized by low charge density, maintains its strength in 10% alcohol solution. On the other hand, the anionic binder of Example 2, characterized by high charge density, yields a much weaker system. However, the anionic binder is proportionately more resistant to the presence of acetone.

TABLE 6
In-Use Strength of Wet Wipes With Cationic or Anionic Binders

| | Binder Composition | | | |
|---|---|---|---|---|
| Organic Solvent in | Example 1 - Cationic | | Example 2 - Anionic | |
| Wetting | MDWT | | MDWT | |
| Composition (4 wt % | (g-force/in | strength | (g-force/in | strength |
| NaCl) | width) | loss* | width) | loss* |
| — | 514 | 0% | 226 | 0% |
| 10 wt % ethanol | 459 | 11% | 137 | 39% |
| 10 wt % iso-propanol | 393 | 24% | 82 | 64% |
| 10 wt % acetone | 388 | 25% | 182 | 19% |

*relative to MDWT for 4 wt % NaCl in pure water

### Example 6

### Wet Wipes In-Use Strength: Effect Of Ethanol Concentration

The fibrous substrates of Example 3 were converted into wet wipes as described in Example 4. The binder composition for all of the fibrous substrates was the cationic binder of Example 1. The wetting compositions were aqueous saline solutions containing 4 wt % NaCl and either no organic solvent or from 10 wt % to 50 wt % ethanol. Add-on of the wetting composition was approximately 300% relative to the weight of the dry substrate. The in-use strength of each type of wet wipe was measured as described in Example 4.

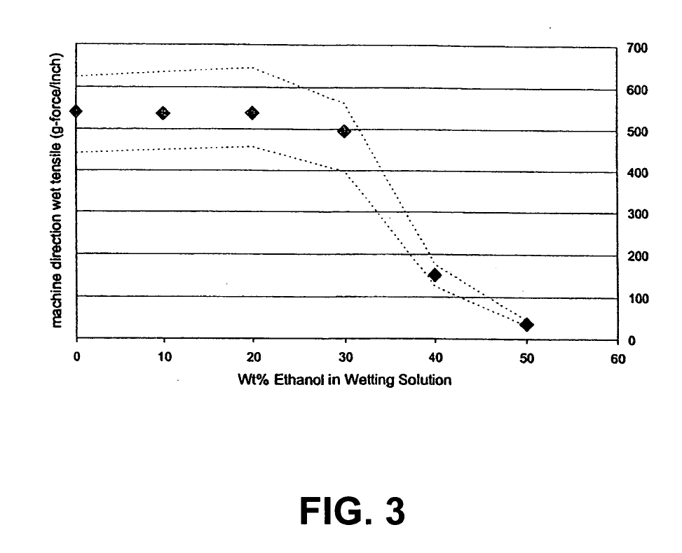

Table 7 lists the measured MDWT values (with standard deviation) for each type of wetting composition. The data from this table are illustrated graphically in FIG. 3. The strength of the cationic binder is maintained in wetting compositions containing up to 30 wt % to 40 wt % ethanol.

TABLE 7
In-Use Strength of Wet Wipes With
Variable Ethanol Concentration

| Wt % Ethanol in | |
|---|---|
| Wetting Composition | MDWT (g-force/inch) |
| 0 | 540 ± 53 |
| 10 | 536 ± 95 |
| 20 | 538 ± 67 |
| 30 | 495 ± 80 |
| 40 | 151 ± 40 |
| 50 | 36 ± 7 |

### Example 7

### Wet Wipes Disposal Strength: Effect Of Salt Concentration

The fibrous substrates of Example 3 were converted into wet wipes as described in Example 4. The binder composition for all of the fibrous substrates was the cationic binder of Example 1. The wetting compositions were aqueous saline solutions containing 30 wt % ethanol and either 1 wt % or 3 wt % NaCl. Add-on of the wetting composition was approximately 300% relative to the weight of the dry substrate.

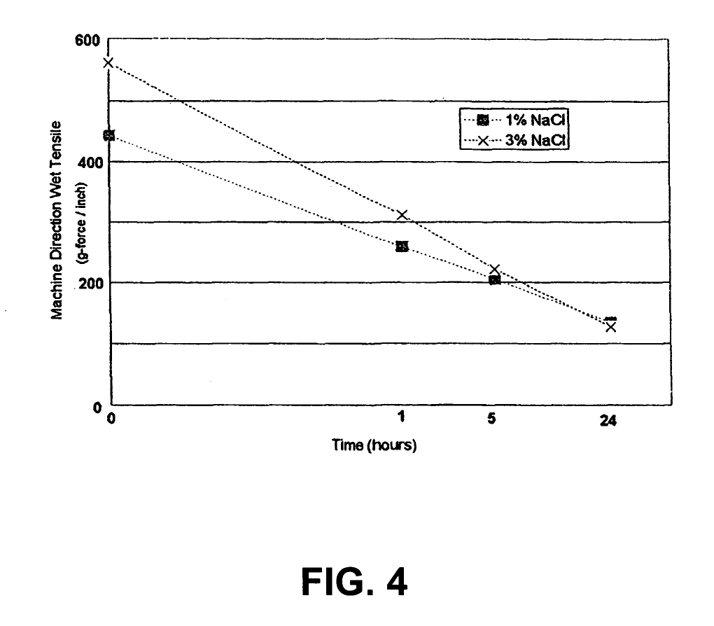

The disposal strength was assessed by transferring a sample into an excess (800 mL) of hard water having a divalent ion concentration of 200 ppm and allowing it to soak for the indicated amount of time before the MDWT was measured. The MDWT values were measured as described in Example 4, and are listed in Table 8. The data from this table are illustrated graphically in FIG. 4. Lower strength in hard water corresponds to improved dispersibility in sewer and septic systems. Wet wipes having a binder composition containing an ionic copolymer can provide useful levels of in-use strength, as a function of NaCl concentration, without sacrificing dispersibility.

TABLE 8
Disposal Strength of Wet Wipes With Variable Salt Concentration

MDWT (g-force/in width)
Salt in Wetting Composition
Containing 30 wt % Ethanol

| Soak time in hard water | | 1 Wt % NaCl | 3 Wt % NaCl |
|---|---|---|---|
| 0 | hours | 443 | 561 |
| 1 | hour | 258 | 311 |
| 5 | hour | 205 | 222 |
| 24 | hours | 134 | 127 |

### Example 8

### Wet Wipes Disposal Strength: Effect Of Wetting Composition Add-On

The fibrous substrates of Example 3 were converted into wet wipes as described in Example 4. The binder composition for all of the fibrous substrates was the cationic binder of Example 1. The wetting compositions were aqueous saline solutions containing 4 wt % NaCl and 30 wt % ethanol. Add-on of the wetting composition relative to the weight of the dry substrate was varied from 50% (damp substrate) to 600% (excess liquid). The disposal strength for each type of wet wipe was measured as described in Example 7.

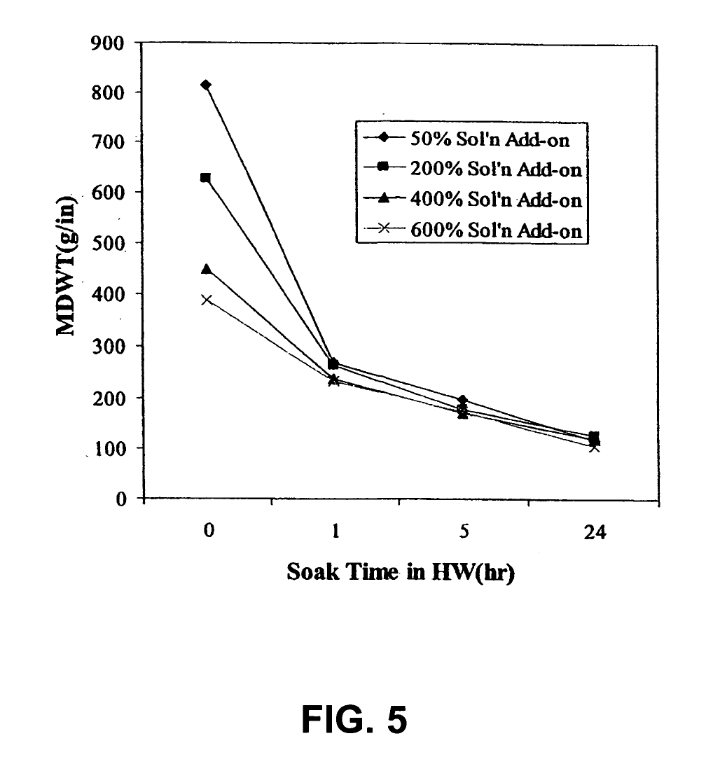

Table 9 lists the measured MDWT values, and the data from this table are illustrated graphically in FIG. 5. Lower initial add-on levels resulted in initially higher strength. When placed in hard water, the strength of the samples degraded, as shown. It is noted that the kinetics of the dispersibility do not appear to change significantly. This consistency of kinetics is advantageous for using wet wipes in a broad range of applications.

TABLE 9
Disposal Strength of Wet Wipes With Variable Salt Concentration

MDWT (g-force/in width)
Wetting Composition Add-On

| Soak time in hard water | | 50% | 200% | 400% | 600% |
|---|---|---|---|---|---|
| 0 | hours | 816 | 626 | 450 | 389 |
| 1 | hour | 269 | 264 | 238 | 233 |
| 5 | hour | 197 | 176 | 169 | 172 |
| 24 | hour | 116 | 126 | 120 | 105 |

It is therefore intended that the foregoing detailed description be regarded as illustrative rather than limiting, and that it be understood that it is the following claims, including all equivalents, that are intended to define the spirit and scope of this invention.

1. A wet wipe, comprising:
fibrous material;
a binder composition for binding the fibrous material into an integral web, the binder composition comprising an ionic copolymer; and
a wetting composition comprising water, a salt, and at least about 10 wt % of an organic solvent;
wherein the binder composition is insoluble in the wetting composition, and the binder composition is dispersible in water containing up to 200 ppm of one or more multivalent ions.

2. The wet wipe of claim 1, wherein the ionic copolymer is the polymerization product of a vinyl-functional cationic monomer and at least one non-ionic vinyl monomer.

3. The wet wipe of claim 2, wherein the vinyl-functional cationic monomer is selected from the group consisting of [2-(acryloxy)ethyl]dimethyl ammonium chloride, [2-(methacryloxy)ethyl]dimethyl ammonium chloride, [2-(acryloxy)ethyl]trimethyl ammonium chloride, [2-(methacryloxy)ethyl]trimethyl ammonium chloride, (3-acrylamidopropyl)trimethyl ammonium chloride, N,N-diallyldimethyl ammonium chloride, [2-(acryloxy)ethyl]dimethylbenzyl ammonium chloride, and [2-(methacryloxy)ethyl]dimethylbenzyl ammonium chloride.

4. The wet wipe of claim 1, wherein the ionic copolymer has the structure: 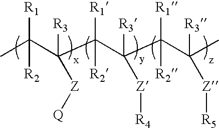
wherein x=1 to about 15 mole percent, y=about 60 to about 99 mole percent, and z=0 to about 30 mole percent;
Q is selected from C₁-C₄ alkyl ammonium, quaternary C₁-C₄ alkyl ammonium, and benzyl ammonium;
Z, Z′ and Z″ are independently selected from —O—, —COO—, —OOC—, —CONH—, and —NHCO—;
R₁, R₁′, R₁″, R₂, R₂′, R₂″, R₃, R₃′ and R₃″ are independently selected from hydrogen and methyl;
R₄ is C₁-C₄ alkyl; and
R₅ is selected from methyl, ethyl, butyl, ethylhexyl, decyl, dodecyl, hydroxyethyl, hydroxypropyl, polyoxyethylene, and polyoxypropylene.

5. The wet wipe of claim 1, wherein the ionic copolymer has the structure: 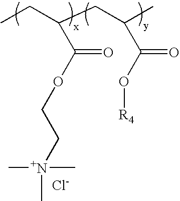
wherein x=1 to about 15 mole percent; y=about 85 to about 99 mole percent; and R₄ is C₁-C₄ alkyl.

6. The wet wipe of claim 1, wherein the ionic copolymer is the polymerization product of at least one non-ionic vinyl monomer and a vinyl-functional anionic monomer selected from the group consisting of acrylic acid, methacrylic acid, itaconic acid, 2-acrylamido-2-methyl-1-propanesulfonic acid (AMPS), 2-methyl-2-propene sulfonic acid, vinyl sulfonic acid, styrene sulfonic acid, 2-sulfopropyl methacrylate, 3-sulfopropyl acrylate, and alkali salts thereof.

7. The wet wipe of claim 1, wherein the ionic copolymer is the polymerization product of a vinyl-functional ionic monomer and at least one non-ionic vinyl monomer; wherein the at least one non-ionic vinyl monomer comprises at least one hydrophobic monomer with an alkyl side chains of 1 to 4 carbon atoms.

8. The wet wipe of claim 1, wherein the ionic copolymer is the polymerization product of a vinyl-functional ionic monomer and at least one non-ionic vinyl monomer; wherein the at least one non-ionic vinyl monomer comprises at least one hydrophobic monomer selected from branched or linear alkyl vinyl ethers, vinyl esters, acrylamides, and acrylates.

9. The wet wipe of claim 1, wherein the wetting composition comprises from about 0.3 wt % to about 10 wt % of at least one salt selected from the group consisting of NaCl, NaBr, KCl, NH₄Cl, Na₂SO₄, ZnCl₂, CaCl₂, MgCl₂, MgSO₄, NaNO₃, and NaSO₄CH₃.

10. The wet wipe of claim 1, wherein the wetting composition comprises from about 10 wt % to about 75 wt % organic solvent.

11. The wet wipe of claim 1, wherein the organic solvent is selected from the group consisting of alcohols, ketones, ethers, acetates, amines, amides, sulfides, sulfoxides, and halogenated hydrocarbons.

12. The wet wipe of claim 1, wherein the organic solvent is selected from the group consisting of ethanol, iso-propanol, and acetone.

13. The wet wipe of claim 1, wherein the wet wipe is not dispersible in the wetting composition, and the wet wipe is dispersible in water containing up to 200 ppm of one or more multivalent ions.

14. A method of making a wet wipe, comprising:
applying a binder composition comprising an ionic copolymer to fibrous material to form a fibrous substrate; and
wetting the fibrous substrate with a wetting composition comprising water, a salt, and at least about 10 wt % of an organic solvent;
wherein the wet wipe is not dispersible in the wetting composition, and the wet wipe is dispersible in water containing up to 200 ppm Ca²⁺ and/or Mg²⁺ ions.

15. The method of claim 14, further comprising drying the fibrous substrate prior to wetting the fibrous substrate.

16. The method of claim 14, further comprising packaging the fibrous substrate as a wipe after drying the fibrous substrate and prior to wetting the fibrous substrate.

17. The method of claim 14, wherein the ionic copolymer is the polymerization product of a vinyl-functional cationic monomer and at least one non-ionic vinyl monomer.

18. The method of claim 17, wherein the vinyl-functional cationic monomer is selected from the group consisting of [2-(acryloxy)ethyl]dimethyl ammonium chloride, [2-(methacryloxy)ethyl]dimethyl ammonium chloride, [2-(acryloxy)ethyl]trimethyl ammonium chloride, [2-(methacryloxy)ethyl]trimethyl ammonium chloride, (3-acrylamidopropyl) trimethyl ammonium chloride, N,N-diallyldimethyl ammonium chloride, [2-(acryloxy)ethyl]dimethylbenzyl ammonium chloride, and [2-(methacryloxy)ethyl]dimethylbenzyl ammonium chloride.

19. The method of claim 14, wherein the ionic copolymer is the polymerization product of at least one non-ionic vinyl monomer and a vinyl-functional anionic monomer selected from the group consisting of acrylic acid, methacrylic acid, itaconic acid, 2-acrylamido-2-methyl-1-propanesulfonic acid (AMPS), 2-methyl-2-propene sulfonic acid, vinyl sulfonic acid, styrene sulfonic acid, 2-sulfopropyl methacrylate, 3-sulfopropyl acrylate, and alkali salts thereof.

20. The method of claim 14, wherein the ionic copolymer is the polymerization product of a vinyl-functional ionic monomer and at least one non-ionic vinyl monomer; wherein the at least one non-ionic vinyl monomer comprises at least one hydrophobic monomer with an alkyl side chains of 1 to 4 carbon atoms.

21. The method of claim 14, wherein the ionic copolymer is the polymerization product of a vinyl-functional ionic monomer and at least one non-ionic vinyl monomer; wherein the at least one non-ionic vinyl monomer comprises at least one hydrophobic monomer selected from branched or linear alkyl vinyl ethers, vinyl esters, acrylamides, and acrylates.

22. The method of claim 14, wherein the wetting composition comprises from about 0.3 wt % to about 10 wt % of at least one salt selected from the group consisting of NaCl, NaBr, KCl, NH₄Cl, Na₂SO₄, ZnCl₂, CaCl₂, MgCl₂, MgSO₄, NaNO₃, and NaSO₄CH₃.

23. The method of claim 14, wherein the wetting composition comprises from about 10 wt % to about 75 wt % organic solvent.

24. The method of claim 14, wherein the organic solvent is selected from the group consisting of alcohols, ketones, ethers, acetates, amines, amides, sulfides, sulfoxides, and halogenated hydrocarbons.

25. The method of claim 14, wherein the organic solvent is selected from the group consisting of ethanol, iso-propanol, and acetone.
